(19) United States
(12) Patent Application Publication
(10) Pub. No.: US 2003/0148386 A1
(43) Pub. Date: Aug. 7, 2003

(54) Method for drug design using comparative molecular field analysis (CoMFA) extended for multi-mode/multi-species ligand binding and disposition

(75) Inventors: Stefan Balaz, Fargo, ND (US); Viera Lukacova, Fargo, ND (US)

Correspondence Address: MUETING, RAASCH & GEBHARDT, P.A., P.O. BOX 581415, MINNEAPOLIS, MN, 55458, US

(73) Assignee: North Dakota State University, Fargo, ND

(21) Appl. No.: 10/292,800

(22) Filed: Nov. 12, 2002

Related U.S. Application Data
(60) Provisional application No. 60/337,349, filed on Nov. 9, 2001.

(51) Int. Cl. G01N 33/53; G06G 7/48; G06G 7/58; G06F 19/00; G01N 33/48; G01N 33/50

(52) U.S. Cl. 435/7.1; 702/19; 703/11

(57) ABSTRACT

An advance in the art of comparative molecular field analysis allows the evaluation of multiple binding modes for the interactions of single or multiple species of ligand molecules with a common macromolecule.

This application claims the benefit of U.S. Provisional Application Serial No. 60/337,349, filed Nov. 9, 2001, which is incorporated herein by reference in its entirety.

### STATEMENT OF GOVERNMENT RIGHTS

This invention was made with government support under grants from the Environmental Protection Agency and the National Institutes of Health, Grant Nos. R82-6652-011 and 1 P20 RR15566-01, respectively. The U.S. Government has certain rights in this invention.

### BACKGROUND OF THE INVENTION

Interactions of ligands with macromolecules are multifarious due to a variety of loosely defined binding sites, at which ligands can bind with varying stoichiometries and orientations. This invention deals with small binding sites that are not much larger than the ligand itself. The local stoichiometry characterizing these sites is 1:1 (one ligand per binding site). It is becoming increasingly apparent that a ligand can often bind in multiple conformations and/or orientations (“binding modes”) at its binding site. See Mattos et al., Multiple Binding Modes. In 3D-QSAR in Drug Design: Theory, Methods, and Applications; Kubinyi, H., Ed.; Escom, Leiden, 1993; pp 226-254 and Lukacova et al, in Hoeltje et al. (eds): Rational Approaches to Drug Design, Prous Science Publ., Barcelona, 2001, pp. 354-358, for reviews of recent x-ray and NMR demonstrations of multiple binding modes of one ligand in one binding site. Multi-mode binding occurs when individual bound ligand molecules representing a single molecular species exhibit different binding modes at a single binding site. Multi-species binding denotes the situation when some of the bound molecules are different molecular species originating by ionization or tautomerism (protomers or tautomers, respectively). With continuing sophistication of methods for structure determination, multiple binding modes are being observed experimentally with increasing frequency (Mattos et al., Multiple Binding Modes. In 3D-QSAR in Drug Design: Theory, Methods, and Applications; Kubinyi, H., Ed.; Escom, Leiden, 1993; pp 226-254; Balaz et al., Multiple Binding Modes in Three-dimensional Quantitative Structure-Activity Relationships. In QSAR in Environmental Sciences; Walker, J., Ed.; Society for Environmental Toxicology and Chemistry, Pensacola (in press); de la Paz et al., Multiple Modes of Binding of Thyroid Hormones and Other lodothyronines to Human Plasma Transthyretin. In The Design of Drugs to Macromolecular Targets; Bedell, C. R., Ed.; John Wiley and Sons, Chichester, 1992; pp 119-172; and Arevalo et al., J. Mol. Biol. 1994, 241, 663-690).

Conceptual 3D-QSAR (Quantitative Structure Activity Relationship) methods use structures and affinities of ligands for binding to a macromolecule to deduce the hypothetical shape and properties of the binding site and to predict affinities of non-tested ligands. If the ligands have a common scaffold, the scaffold conformations or orientations may vary among the bound ligands even if each ligand only has one binding mode. (Mattos et al., Nat. Struct. Biol. 1994, 1, 55-58.) A 3D-QSAR analysis starts with an alignment of ligands or their placement in a putative binding site. Ambiguousness of this step increases with ligand flexibility. Among many subjective options, the most common starting point is ligand alignment, which utilizes either an atom-based or property-based superposition of ligands according to a pharmacophore hypothesis (Lemmen et al., Persp. Drug Discov. Des. 2000, 20, 43-62). The alignments are evaluated one at a time on the basis of statistical indices and predictive ability of a 3D-QSAR analyses. Occasionally, alternate binding modes are employed for the worst-fitting ligands (Diana et al., J. Med. Chem. 1992, 35, 1002-1008; Oprea et al., J. Med. Chem. 1994, 37, 2206-2215; Nicklaus et al., J. Comp. Aided Mol. Des. 1992, 6, 487-504; and Klebe et al., J. Med. Chem. 1993, 36, 70-80).

The importance of multiple binding modes and its threat to the formulation of meaningful analyses are well known in the area of drug design. Some pharmacophore-mapping methods (Martin et al., J. Comput. Aided Mol. Des. 1993, 7(1):83-102) consider alternative binding modes of active/inactive ligands to construct a spatial representation of the receptor. In the area of 3D-QSAR, the approaches of Crippen's group (Crippen, J. Med. Chem. 1979, 22(8) 988-997) evaluate alternative binding modes for each ligand in the search for acceptable description of the binding data and the corresponding spatial representation of the receptor. Although still selecting one binding mode for each ligand, these approaches at least reduce the subjective input to the construction of the representation of the binding site.

Comparative molecular field analysis (CoMFA) (U.S. Pat. No. 5,307,287, Cramer, III et al.) is a 3D-QSAR method that has been widely used to predict the three-dimensional structure of a binding site on a receptor protein (or other biomolecule) where the three-dimensional structure of the protein is unknown. A series of ligands (typically between 10 and 100) that bind to the binding site is computationally analyzed. The analysis correlates experimentally determined binding affinities for each of the ligands with the differences in the shapes of their steric, electronic and often also hydrophobic fields. The procedure characterizes the hypothetical binding site by coefficients assigned to the probe/ligand interaction energies (electrostatic, steric, sometimes hydrophobic fields) in individual points of a grid encompassing the aligned ligands. CoMFA allows the researcher to predict the likely binding behavior of a molecule not included in the initial data set. It also allows immediate computational testing of proposed molecular modifications to a ligand, thereby facilitating rational design of receptor inhibitors, agonists, antagonists, effectors and other drugs.

CoMFA, as originally developed, assumes each ligand binds in a single mode. That is, when bound to the receptor at the binding site, the ligand is assumed to be present in only one three-dimensional conformation, orientation, and state (due to ionization or tautomerism). However, as noted above, recent experimental evidence suggests that some compounds may bind to a receptor protein in more than one mode. Because conventional CoMFA can accommodate only one binding mode per ligand, the preferred binding mode must be pre-identified by the researcher. It can be difficult to identify the preferred binding mode for a ligand, and for expediency the same binding mode is frequently selected for all ligands in the set. If the resulting analysis does not statistically account for a sufficient amount of the variation among the ligands, a time-consuming iterative process is initiated wherein the researcher repeatedly identifies and tests different binding modes for outliers in an effort to improve the analysis.

A brute-force examination of alternate binding modes of all ligands leads to a combinatorial explosion in the number of required 3D-QSAR analyses: M₁×M₂×. . . ×M_{L} analyses are needed for L ligands, if the i^{th} ligand binds in Mᵢ binding modes. To illustrate the magnitude of this number, let us consider a small set of 30 ligands, each binding in two modes. A systematic evaluation requires 2³⁰ one-mode 3D-QSAR analyses, which would be done in about 34 years by a fast method consuming only I second per analysis. Apparently, an exhaustive one-by-one evaluation of possible modes for all ligands is practically impossible for any real-world QSAR problem.

Theoretically, the combinatorial problem could be bypassed by a 3D-QSAR method that would conceptually treat multiple binding modes. The method would need to provide mode prevalencies, in addition to standard outputs comprising the correlation equation and a spatial model of the binding site. The use of such a method is necessary if multiple binding modes are experimentally observed or expected for some ligands in the studied set. The method would be very useful even if one mode prevails for each ligand because it would perform efficient mode selection by setting the optimized prevalencies of improbable modes close to zero.

Researchers have attempted to modify CoMFA to accommodate multiple binding modes (Cramer III et al., J. Am. Chem. Soc. 1988, 110, 5959-5967). Multi-mode ligand binding is represented by a field of interaction energies that are obtained as a weighted average of the energies for individual modes, with either identical (Nicklaus et al., J. Comp. Aided Mol. Des. 1992, 6, 487-504; and Kim et al., J. Med. Chem. 1991, 34, 2056-2060) or different (Cramer III et al., U.S. Pat. No. 5,307,287, 1994) weights corresponding to expected mode prevalencies. In effect, multi-mode CoMFA is a single mode analysis, but using a weighted average of the relevant probe/ligand interaction energies in the grid points. The “weighted field approach” is, for example, implemented in the current version of SYBYL (v. 6.8, Tripos Inc., St. Louis Mo.). This analysis, however, contradicts the thermodynamic premise that the overall association constant is a sum of the partial association constants for individual modes. Moreover, for reasons described below, the weighted field approach requires pre-identification of the various binding modes as well as their prevalencies, which are very difficult if not impossible to predict. As a result, this method is unsound and does not lead to reliable results.

4D-QSAR analysis (Hopfinger et al., J. Am. Chem. Soc. 1997, 119, 10509-10524) relies on ensemble averaging to incorporate conformational flexibility and alignment freedom. The averaging may suffer from the same problem as the field weighting in CoMFA. Many ligand conformations are generated by molecular dynamics and placed into the grid in different alignments. Occupancies of individual grid points are used as descriptors in the analysis that includes partial least squares (PLS) and genetic algorithms. The result is a manifold of 3D-QSAR models.

Functions similar to Boltzmann probabilities were used in pseudo-receptor models considering different conformations (Vedani et al., Altex 1999, 16, 142-145; and Vedani et al., Quant. Struct. Act. Relat. 2000, 19, 149-161) and protonation states of ligands (Vedani et al. J. Med. Chem. 2000, 43, 4416-4427). Interestingly, no proton affinities of individual ligands were needed in the latter analysis. The approach optimizes the atom composition in a multi-atom envelope representing the pseudo-receptor by a genetic algorithm with crossovers and transcription errors. The result is a fuzzy family of several hundred models, which are visualized using most frequently occurring atom types. The frequencies of individual atoms were not given but probably are rather low.

There is a need for an improved method for computationally analyzing structure-activity relationships between ligands and macromolecules, which takes into consideration the multiple binding modes of ligands.

### SUMMARY OF THE INVENTION

It has proved computationally problematic to accommodate multiple binding modes in 3D-QSAR analyses, for example using comparative molecular field analysis (CoMFA). The present invention provides an advance in the art of computational biology by making it possible to incorporate multiple ligand binding modes into a single CoMFA analysis. Like conventional CoMFA (U.S. Pat. No. 5,307,287), the analysis involves calculation of molecular force fields, alignment of the ligands and field fit, and graphical display of the results. However, in the present method, it is not necessary to pre-identify the prevalencies of individual binding modes. As one of the results, the procedure will determine the statistical distribution of the prevalencies of the various modes and effectively eliminate improbable binding modes. Therefore, the procedure can be used to find realistic binding modes in a larger set of input modes. This capability removes the bottleneck for performing CoMFA analyses (i.e., the researcher's educated guess of one probable binding mode for each ligand) and allows for automation of the whole process.

In addition, the output of the method of the invention includes a three-dimensional map of the binding site that is similar in appearance to that provided by single-mode CoMFA. However, the map will provide a more realistic picture of actual binding site because the modeling procedure depicts the underlining events in a more realistic way.

The present invention thus provides a computer-based method for generating a three-dimensional quantitative structure activity relationship of a series of ligand molecules in association with a common macromolecule. The method includes one or more of the following steps:

identifying one or more binding modes j for each ligand molecule i in the series of ligand molecules;

placing each binding mode j for each ligand molecule i in said series into a grid for calculation of binding energies;

determining, in a multiplicity of grid points k for each binding mode j of each ligand molecule i, the interaction energy Xᵢⱼₖ of binding mode j with a selected probe;

expressing an association constant Kᵢ for each ligand molecule i as a nonlinear function of the interaction energies Xᵢⱼₖ for each binding mode j to yield a nonlinear binding function;

optimizing the regression coefficients in the nonlinear binding function;

linearizing the nonlinear binding function to allow the use of partial least squares for iterative optimization of at least one regression coefficient to yield a linearized correlation function;

applying a partial least squares procedure repetitively to the linearized correlation function until self-consistency to correlate the observed biological activity data with the interaction energies Xᵢⱼₖ of the ligand molecules; and

calculating the optimized distribution of prevalencies of individual binding modes using the ratio of partial association constant Kᵢⱼ and the association constant Kᵢ for each ligand molecule i with all the other ligand molecules in the series.

Binding modes j can include different conformations or orientations or both. Linearization is used when the number of variables is greater than the number of ligands; otherwise, the linearization steps can be omitted.

In some embodiments, the method also includes visualizing the three-dimensional quantitative structure activity relationship. Typically, visualization can be achieved by using computer graphics to display the correlation among the ligand molecules in the series.

In some embodiments, the method further includes, after linearizing the binding function but before applying the partial least squares procedure, linearizing a nonlinear disposition function containing at least one variable describing a property of each ligand molecule i. Possible properties include lipophilicity, amphiphilicity, acidity, reactivity, 3-dimensional shape of the ligand molecules, and the time of exposure to the common macromolecule. This allows the use of partial least squares for iterative optimization of at least one regression coefficient to yield a linearized disposition function.

Applying the partial least squares procedure can include applying a partial least squares procedure repetitively to the linearized correlation function, the linearized disposition function, and/or a mathematical combination of the linearized correlation and disposition functions until self-consistency to correlate the observed biological activity data with the interaction energies Xᵢⱼₖ and/or properties of the ligand molecules.

Optimizing the regression coefficients can include employing a strategy such as forward selection, backward selection or the use of a genetic algorithm.

The method of the invention can be used to analyze multi-species binding. When at least one ligand molecule is characterized by a plurality of species that originate by ionization or tautomerism, the method preferably includes determining, in a multiplicity of grid points k for each binding mode j for each species of each ligand molecule i, the interaction energy Xᵢⱼₖ of binding mode j with a selected probe.

### BRIEF DESCRIPTION OF THE DRAWINGS

FIG. 1 is a flow chart illustrating a forward-selection procedure for optimization of regression coefficients in Eq. (5) iteratively using linearized Eq. (7). Outlining, Shaping, and Detailing are three phases of the optimization.

FIG. 2 illustrates PCDF congeners (compound 29, Table 1 shown) in two, four and sixteen modes. The PCDF congeners have carbons 1, 4, 6, 9 superimposed to carbons 1, 4, 6, 9 of TCDD (thin lines) in different combinations. Mode A corresponds to the IUPAC nomenclature, other modes are obtained from the standard orientation (A) by flipping around the y-axis (mode B), x-axis (mode D), and both x- and y-axes (mode C). To obtain the sixteen modes, each of the modes A-D had the alignment modified by shifting the PCDF molecule, in the plane of the skeletons, to the top (T), the left side (L) or the top left corner (TL) of the box enclosing the TCDD molecule (for details see FIG. 3). TCDD structure (thin lines) and skeleton numbering is also shown.

FIG. 3 shows that sixteen binding modes were generated by translation of existing four binding modes A-D (mode A for compound 8 in Table 1 shown in yellow) in the plane of the skeletons to obtain the overlap of the van der Waals surfaces on the top (A-T), left (A-L), and both left and top (A-TL) sides of TCDD molecule (blue).

FIG. 4 shows alignments of a PCDF congener (13, Table 1, thin lines) to the TCDD molecule (cylinders) in one (A), two (B), four (C), and sixteen modes (D). The alignments A-C are based on superposition of skeleton carbons 1, 4, 6, and 9 in different orientations (FIG. 2). In the alignment D, the PCDF molecules are matched with the top and left parts of the TCDD molecule.

FIG. 5 is a graph of R² versus number of components, showing quality of the fit vs. the number of components for correlations obtained in different iterations for 16 binding modes. A: 36 variables, iteration 8 (●), iteration 30 (◯), and iteration 42 (▴). B: iteration with best quality of the fit for 6 variables (●), 24 variables (◯), and 36 variables (▴).

FIG. 6 is a graph showing improvement of the fit (R²-●) and predictions (q²-◯) of the correlation with 16 binding modes with addition of variables in the second phase (Shaping—FIG. 1). The shown q² values are valid for the sequence that led to the best correlation in the second phase. The data for the six variables represent the results of the first phase (Outlining).

FIG. 7 is a graph showing development of R² (●) and q² (◯) of the correlation with 16 binding modes during the third phase (Detailing—FIG. 1). Addition of more columns did not improve the correlation. The stepwise removal of columns led to increase of predictive ability of the correlation. The best correlations for the given numbers of variables are shown. The correlation with 22 variables was selected as final.

FIG. 8 is a graph showing calculated and predicted affinities versus experimental affinities for the correlations considering one binding mode (◯) and sixteen binding modes (●). Predicted binding affinities are for compounds in the test data set using the model developed from compounds in training data set.

FIG. 9 shows comparison of electrostatic (left) and steric (right) properties of the receptor binding site estimated by one-mode CoMFA analysis (A) and the multi-mode approach (B-two modes, C-four modes, and D-16 modes) for the CH₃⁺ probe. In electrostatic fields, favorable regions are colored cyan and unfavorable regions red. In steric fields, favorable regions are green and unfavorable regions are yellow. The best binding PCDF congener (29, Table 1, mode A for multi-mode CoMFA) is shown.

FIG. 10 is a graph showing comparison of input and output distribution of prevalencies of individual binding modes for the weighted-field CoMFA (Sybyl, version 6.7. Tripos Inc., St. Louis, Mo., USA, 2001). The results from the four-mode correlation were used as input distribution. Modes: A (●), B (◯), C (▴) and D (∇). Identity line shown.

FIG. 11 is a graph showing calculated (●) and predicted (◯) affinities versus experimental affinities for 1, 2, 4 and 16 binding modes.

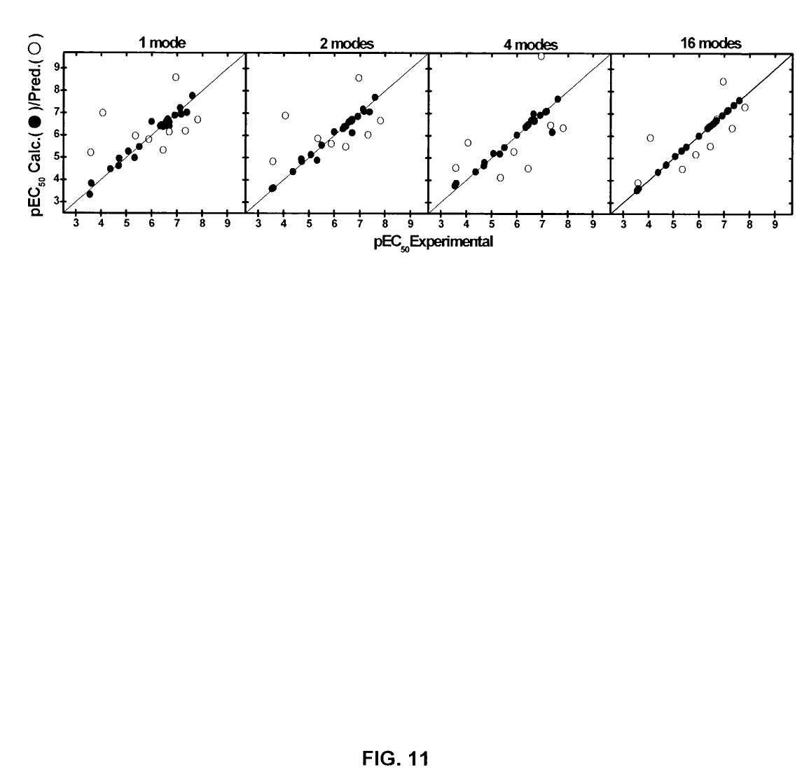

### DETAILED DESCRIPTION OF ILLUSTRATIVE EMBODIMENTS

Definitions

A “ligand” is a low-molecular-weight molecular species that associates with a macromolecule at a binding site. Although intermolecular interactions between ligands and macromolecules can be mediated by covalent or noncovalent interactions, the method of the present invention is useful for analyzing noncovalent interactions. Noncovalent interactions include, for example, ionic interactions, hydrophobic interactions, van der Waals interactions, and hydrogen bonding. The term “binding site” refers to a site on the macromolecule to which the ligands bind. Typically, the binding site is a pocket or cavity of a macromolecule that is not much larger than interacting ligands. The “(binding) mode” denotes a specific conformation and/or orientation of a ligand molecule in the binding site. The terms “binding in multiple modes” or “multi-mode binding” refer to the situation when a ligand binds to a binding site of a macromolecule in more than one conformation or orientation, such that individual bound ligand molecules, although they constitute a single molecular species, exhibit different binding modes. “Multi-species binding” denotes the situation when some of the bound molecules are different molecular species originating by ionization or tautomerism (protomers or tautomers, respectively).

The binding site on the macromolecule is small so that the local stoichiometry of the complex is strictly 1:1. If individual bound molecules of the ligand were depicted simultaneously, the structures would, at least partially, overlap due to the limited size of the binding site. The ratio of the number of ligand molecules bound in the given mode to the total number of bound ligand molecules is denoted as the mode prevalence. Free energies of binding do not differ sufficiently for individual binding modes to warrant dominance of a single mode; rather, they determine the mode prevalencies via the Boltzmann probabilities.

The method is used to computationally analyze a series of ligands for which experimental binding affinities are available. The analysis starts with an alignment of ligands or their placement in a putative binding site. Ambiguousness of this step increases with ligand flexibility. Among many subjective options, the most common starting points are ligand alignments, which utilize either an atom-based or property-based superposition according to a pharmacophore hypothesis (Lemmen et al., Persp. Drug Discov. Design 2000, 20, 43-62). See also U.S. Pat. No. 5,307,287.

Observed Association Constant of a Multi-Mode Ligand Binding

For practical reasons, usually the total drug-receptor association constant Kᵢ is only determined in experimental studies and no attempt is made to examine the binding of individual modes. The relation between Kᵢ and the partial association constants Kᵢⱼ characterizing individual binding modes can be found by kinetic and thermodynamic analyses of the multi-mode ligand/receptor interaction (Jullien et al., J. Chem. Edu. 1998, 75, 194-199; Wang et al., J. Mol. Biol. 1995, 253, 473-492; Balaz et al., Chemometr. Intell. Lab. Sys. 1994, 24, 185-191; Hornak et al., Quant. Struct. Act. Relat. 1998, 17, 427-436; and Smith et al., Chemical Reaction Equilibrium Analysis: Theory and Algorithms; John Wiley and Sons, New York, 1982). Schematically, the fast and reversible 1:1 interaction of the receptor R with the i^{th} ligand Lᵢ that binds in m binding modes is depicted as

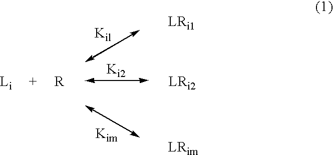

The binding of the ligand in individual modes in the binding site is characterized by partial association constants Kᵢⱼ (j=1, 2, . . . m). The total concentration of receptor/ligand complex is equal to the sum of concentrations of the individual complexes in which the ligand binds in different binding modes. The observed overall association constant can then be expressed as (Balaz et al., Chemometr. Intell. Lab. Sys. 1994, 24, 185-191)

$${K}_{i} = \frac{[ {LR}_{i} ]}{[ {L}_{i} ] \times [ R ]} = \frac{[ {LR}_{i1} ] + [ {LR}_{i2} ] + … + [ {LR}_{im} ]}{[ {L}_{i} ] \times [ R ]} = {\sum }_{j = 1}^{m} {K}_{ij} \qquad ( 2 )$$

The simple Eq.(2) is in accord with published analyses of formally analogous situations: the statistical thermodynamic (Wang et al., J. Mol. Biol. 1995, 253, 473-492) and equilibrium (Balaz et al., Chemometr. Intell. Lab. Sys. 1994, 24, 185-191; and Hornak et al., Quant. Struct. Act. Relat. 1998, 17, 427-436) treatment of multi-mode binding in ligand/protein interactions and kinetic analyses of a reversible uni-molecular reaction leading to different products (Jullien et al., J. Chem. Edu. 1998, 75, 194-199) or isomers (Smith et al., Chemical Reaction Equilibrium Analysis: Theory and Algorithms; John Wiley and Sons, New York, 1982). Eq. (2) represents the basis for incorporation of multiple binding modes into any conceptual 3D-QSAR method. It should be noted that the ligand molecules in scheme (1) represent one molecular species, the nonionized molecules. The molecules can be present in different conformations in the solution surrounding the binding site. However, the conformations are assumed to adapt quickly upon binding.

Similar analysis as outlined in Eq. (1) and Eq. (2) can be done to evaluate binding of populations of s species of ligand molecules that can originate by ionization or tautomerism, each species binding in m binding modes. The observed overall association constant is then

$${K}_{i} = {\sum }_{j = 1}^{s} {f}_{ij} \times {\sum }_{k = 1}^{m} {K}_{ijk} \qquad ( 3 )$$

where fᵢⱼ is the fraction of the j^{th} molecular species in the i^{th} compound with respect to the concentration of free molecular species. The fractions f can be obtained from the definitions of the equilibrium constants for ionization and/or tautomerism for the given experimental conditions. The values of f are usually known and need not be optimized. As follows from Eq. (3), additivity of the partial association constants cannot be used in multi-species 3D-QSAR correlations as recently suggested. (Vedani et al., J. Med. Chem. 2000, 43, 4416-4427.)

Multiple Binding Modes in CoMFA

The association constant for a ligand binding in a particular binding mode is correlated to the ligand/probe interaction energies in CoMFA as (U.S. Pat. No. 5,307,287, Cramer, III et al.):

$${K}_{ij} = exp ( {C}_{0} - {C}_{e} \times {E}_{ij} + {\sum }_{k = 1}^{f \times g} {C}_{k} \times {X}_{ijk} ) \qquad ( 4 )$$

The summation goes through f×g independent variables, where f is the number of used fields (steric, electrostatic, occasionally hydrophobic) and g is the number of the used grid points. The independent variables, Xᵢⱼₖ, are energies of interaction between a probe placed in the k^{th} grid point and the i^{th} ligand molecule in the j^{th} binding mode. The regression coefficients Cₖ characterize significance of field contributions in each grid point for overall binding. Conformational energy Eᵢⱼ, although seldom used in the one-mode CoMFA analysis, can be of importance when several binding modes are considered. (In Example I, however, no conformational energy term was needed because the studied compounds are completely rigid.)

Standard one-mode CoMFA uses logarithmized Eq. (4) that is linear in the optimized regression coefficients C. For multi-mode ligand binding, the correlation equation of the observed association constant Kᵢ with the probe/ligand interaction energies Xᵢⱼₖ results from combination of Eqs. (2) and (4):

$${K}_{i} = {\sum }_{j = 1}^{m} exp ( {C}_{0} - {C}_{e} \times {E}_{ij} + {\sum }_{k = 1}^{f \times g} {C}_{k} \times {X}_{ijk} ) \qquad ( 5 )$$

The j-summation goes through m binding modes. Eq. (5) is nonlinear in regression coefficients C. For simplicity, the invention is described in detail below using a multi-mode binding analysis, as described by Eq. (5), that assumes that each ligand in the series is a single species (i.e., using an analysis that does not take into account protomers or tautomers). Adaptation of the results for multi-species binding is straightforward: the CoMFA equation, similar to Eq. (5), would result from combinations of Eqs. (3) and (4).

Linearization of Optimized Function in CoMFA

The number (f×g plus) of optimized coefficients C is usually much higher than the number of tested compounds, mathematically complicating the task of optimizing the coefficients. Single-mode CoMFA thus uses a special procedure, partial least squares analyses—PLS (described in U.S. Pat. No. 5,307,287, Cramer, III et al.), to optimize the coefficients. PLS requires that the underlying equation is linear in optimized coefficients. Conveniently, the binding function for single mode CoMFA (Eq. (4)) is linear.

For ligands with multiple binding modes, on the other hand, the observed association constant can be described as the sum of the partial association constants of the individual binding modes. When this relation is applied to CoMFA, the resulting binding function is non-linear in optimized coefficients, and PLS cannot be utilized. Multi-mode CoMFA as provided by the invention is made possible by the linearization of the non-linear binding function which, in turn, enables PLS to be used for coefficient optimization. Linearization of the multi-mode binding function is the core of invention.

In order to use PLS, it is Eq. (5) that needs to be linearized. One of the possibilities is to use the first two terms in the Taylor expansion of the exponentials in Eq. (5) as exp(x)≅exp(M)×(1+x−M) for x approaching M. Each exponential in Eq. (5) represents a Kᵢⱼ and can be expanded around the number M=InKᵢⱼ without introducing much error (less than 10% if the exponent is from the interval <InKᵢⱼ −0.5; InKᵢⱼ+0.5>):

$${K}_{i} = {\sum }_{j = 1}^{m} {K}_{ij} \times ( 1 - ln {K}_{ij} + {C}_{0} - {C}_{e} \times {E}_{ij} + {\sum }_{k = 1}^{f \times g} {C}_{k} \times {X}_{ijk} ) \qquad ( 6 )$$

Separation of variables and normalization of Eq. (6) through division by the observed association constants Kᵢ to bring all variables to comparable ranges result in

$$1 - {\sum }_{j = 1}^{m} \frac{{K}_{ij}}{{K}_{i}} + {\sum }_{j = 1}^{m} \frac{{K}_{ij}}{{K}_{i}} \times ln {K}_{ij} = {\sum }_{k = 1}^{f \times g} {C}_{k} \times {\sum }_{j = 1}^{m} {X}_{ijk} \times \frac{{K}_{ij}}{{K}_{i}} + {C}_{0} \times {\sum }_{j = 1}^{m} \frac{{K}_{ij}}{{K}_{i}} \qquad ( 7 )$$

Eq. (7) is the final equation that is used for iterative optimization of the regression coefficients C. The partial association constants Kᵢⱼ are initially unknown and depend on the regression coefficients C as is apparent from Eq. (4). The PLS procedure needs to be applied iteratively, starting with suitable initial estimates of either Kᵢⱼ or C (the latter option was used in this study). Once C are optimized, new Kᵢⱼ from Eq. (4) can be calculated and the procedure repeated until self-consistency.

In Eq. (5), all the optimized regression coefficients C are contained in the linear form in the exponent of the exponential function exp(x) that is used to derive Eq. 6. Therefore, it was possible to perform the Taylor expansion in the simplified way as shown in the paragraph above Eq. (6). The same solution can be obtained by a rigorous Taylor expansion of Eq. (5), describing the observed association constant Kᵢ as a function of the regression coefficients C, around the values C that represent either the initial estimates or the results of the previous iteration.

Optimization of Regression Coefficients

Good initial estimates of the regression coefficients C are very important for successful optimization. An exhaustive search is impossible due to the large number of the coefficients C, therefore an efficient sampling strategy had to be deployed. In general, variable selection is achieved by either choosing the informative variables or discarding redundant variables in step-wise forward-selection and backward-selection ways or their combinations. Suitable procedures include principal component analysis, partial least squares analysis, Procrustes analysis, genetic algorithms, neural networks, and their combinations (Guo et al., Chemomet. Intelligent Lab. Sys. 2002, 61, 123-132 and the references cited therein).

It should be noted that in instances where the number of variables is less than the number of compounds, the regression coefficient can be optimized directly by the non-linear regression analysis.

In the Example I, a gradual increase in the number of independent variables Xᵢⱼₖ is used. It should be appreciated that similar results as in the Example I can be achieved by any variable selection method. The used forward-selection procedure can navigate the solutions into the regions of the parameter space that provide a realistic picture of the binding site. As long as the number of the optimized coefficients Cₖ stays below the number of ligands, the model is deterministic and has a higher chance to arrive at realistic values of Cₖ than later in the optimization process when the number of variables exceeds the number of ligands and the PLS procedure selects some of the infinite number of solutions. By gradual addition or deletion of variables, a satisfactory correlation equation is constructed utilizing a lower number of variables than in one-mode CoMFA. The forward-selection procedure could possibly improve the realism of the description in the one-mode CoMFA analysis.

Selection of variables to be added to the correlation equation is done with the aim to maintain the maximum amount of information, while minimizing the number of variables. This intent leads to three basic criteria for variable selection: (1) high and sustained variability, (2) a minimal number of collinear variables carrying similar information, and (3) more or less even distribution of selected grid points around the aligned molecules.

Variability of independent variables Xᵢⱼₖ is usually characterized by the standard deviation (SD). The SD values, however, do not uncover the situations when variability is high due to one or two extreme Xᵢⱼₖ values that substantially differ from the rest. Technically, this situation is encountered when there are only one or two ligands in the series that differ from the rest of aligned ligand molecules in certain part of the 3D-space. These singularities lead to correlations that lack physical meaning. The leave-one-out cross-validation can effectively exclude the correlations with singularities if there is only one extreme Xᵢⱼₖ value per variable. The identification of the correlations with singularities becomes more difficult for two or three extreme Xᵢⱼₖ values per variable. The singularity problem might be less severe in one-mode CoMFA because of a high number of variables that are included in the final correlation. In multi-mode CoMFA, the forward-selection procedure could pick the variables with singularities and use them as indicator variables to account for the binding differences of respective ligands. Sustained variability was checked for variables exhibiting a large difference between the median and the mean values.

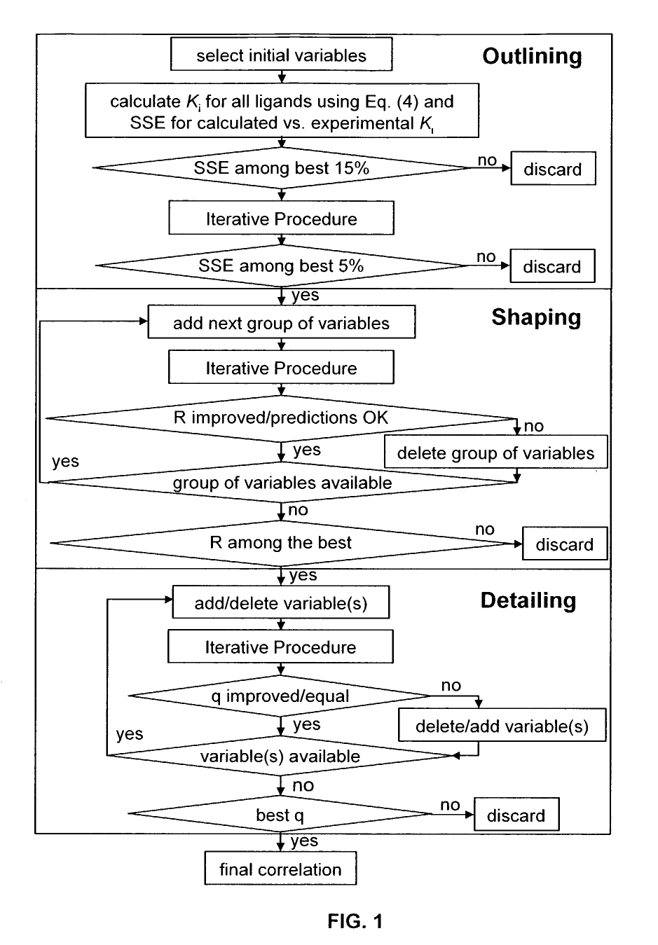

The Smart Region Definition (SRD) procedure in GOLPE (Pastor et al., J. Med. Chem. 1997, 40, 1455-1464) facilitates the selection of variables based on the described criteria. Forward-selection of variables is done in three phases, which we call Outlining, Shaping, and Detailing (FIG. 1).

As applied in Example 1, the first phase, Outlining, used only six variables: three steric and three electrostatic interaction energies. The low number of variables allowed a complete brute-force evaluation of a large number of initial estimate sets within reasonable time. All plus/minus combinations of numbers 0.1, 0.2, . . . , 1.0 were examined as initial estimates for the six regression coefficients Cₖ by calculation of the total binding constants using Eq. (5). The results were ranked using the sum of squares of errors (SSE) of the linear correlation with the unity slope and optimized intercept between calculated and experimental binding affinities. The 15% of estimates with the lowest SSE values were optimized by the Iterative Procedure. The best C sets (5%) with the lowest sum of squares of errors (SSE) between observed and calculated Kᵢ advanced to the second phase (Shaping). Alternatively, the Outlining can be started using initial estimates of individual mode prevalencies Kᵢⱼ/Kᵢ. In this case, the variables of the linearized Eq. (7) need to be calculated and the regression coefficients Cₖ in Eq. (7) obtained by PLS.

In the second phase, Shaping, the best sets of coefficients from Outlining are optimized and the dataset was gradually expanded by addition of groups of variables. In Example I, a group consisted of 6 variables in the first step and 12 variables in subsequent steps in this case. For the added variables, zeros are used as the initial estimates of the regression coefficient and all coefficients are optimized by the Iterative Procedure. Combinations of several groups of variables are examined as shown in the flow-chart (FIG. 1) with the aim to minimize SSE. This phase provided several dozens of correlations with low SSE values.

The third phase, Detailing, aims at fine-tuning of the correlation by addition of small groups (2-6) of variables. The groups are formed by regrouping the variables that were rejected in the second phase. The best correlations from the previous phase are optimized by the Iterative Procedure starting with zero as the initial estimate for the added variable. Since SSE exhibit minimal differences at this stage, the criterion for acceptance of the variable is the cross-validated correlation coefficient q from the leave-one-out procedure. After the maximum q is reached, the correlation is further amended by deletion of variables (one at a time) that had the least contribution (the lowest standard deviation in the column Cₖ×Xᵢⱼₖ) to the calculated affinity. The deletion is accepted if there was no substantial impact on the q value.

Iterative Procedure

An iterative procedure is used to optimize the nonlinear regression coefficients Cₖ in Eq. (5) using the linearized Eq. (7). Each iteration in the Iterative Procedure includes four steps: (i) selection of the set of regression coefficients Cₖ, which can either be initial estimates or come from the previous run; (ii) calculation of the partial association constants Kᵢⱼ using Eq. (4); (iii) calculation of the variables of the linearized Eq. (7); and (iv) calculation of the regression coefficients Cₖ in Eq. (7) by PLS. In each iteration, PLS correlation equations with the number of components ranging from one to the maximum (the number of included variables or the number of compounds in the set, whichever is smaller) are derived. The correlation with the best quality of the fit is selected and its regression coefficients are used as initial estimates for next iteration. Quality of the fit is assessed using the sum of squares of errors (SSE) between calculated and observed Kᵢ.

The iterative procedure stops when predetermined conditions are achieved. In Example I, the iterative procedure stopped when the change in SSE was lower than 0.5% in 15 consecutive iterations or when the maximum number of 50 iterations was reached, whatever occurred earlier. Among correlation equations formed in individual iterations, the best equations for a particular set of descriptor variables are then chosen based on the lowest SSE between calculated and observed Kᵢ values. The best equations are statistically characterized by calculation of the correlation coefficient (R), the predictive sum of squares of deviations (PRESS), and q based on the leave-one-out cross-validation with the optimal number of components. The best correlation for the given set of variables has either the lowest SSE in Shaping or the lowest SSE and highest q in the Detailing. In Example I, if several models with comparable SSE and q values were produced, the model with the best predictions for the compounds with semiquantitative binding affinities (compounds 1, 4 and 15 in Table 1) was selected.

Disposition Function

Biological activity in organisms and other complex systems is a consequence of both the molecule's ability to get to the receptor site and the molecule's ability to bind to the receptor (the association constant Kᵢ or “binding function”). A molecule's ability to penetrate to the receptor site can be described by the disposition function A(pᵢ,t) with physicochemical properties pi and the exposure time t as variables. The disposition function may include, for example, one or more variables associated with lipophilicity, amphiphilicity, reactivity, acidity, 3-dimensional shape of the molecules, and the time of exposure. An example of the disposition function is (Balaz and Lukacova, J. Mol. Graphics Model. 2002, 20: 479-490).

$$A ( {p}_{i} , t ) = \frac{1}{{AP}^{β} + B} {ⅇ}^{- \frac{{CP}^{β} + D}{{AP}^{β} + B} t} \qquad ( 8 )$$

Here, c is the ligand concentration in the surroundings of the binding site, c₀ is the total ligand concentration in the system. The terms A, B, C, and D describe membrane accumulation, distribution in the aqueous phases, and lipophilicity-dependent and -independent metabolism, respectively. Lipophilicity is described by the reference 1-octanol/water partition coefficient P. The empirical coefficient β is optimized by regression analysis, along with further regression coefficients. For ligands that do not ionize or ionize to the same degree, the regression coefficients are the terms A-D. Otherwise, the terms A-D are functions of acidity and other variables. For compounds which ionize to the degree (s-1), where s is the total number of molecular species, the terms A, B, C, D (Y) from Eq. (8) can be expanded as (Balaz et al., J. Theor. Biol. 178 (1996) 7-16):

$$Y = {Y}_{0} + {\sum }_{j = 1}^{s - 1} {Y}_{j} \times {10}^{ sgn \times {\sum }_{k = 1}^{j} {pK}_{jk}} \qquad ( 9 )$$

The subscripts 0 and j indicate the quantities associated with non-ionized molecules and with the species ionized to the j^{th} degree, respectively. The term sgn=1 for bases and sgn=−1 for acids (like the sign of the charge of the resulting ion). Sometimes in vitro biological testing is performed under the conditions of varying acidity of the external medium, pHₑ. In this case, the terms Yⱼ associated with the molecular species ionized to the jth degree in Eq. (9) can be further deconvoluted (Balaz et al., J. Theor. Biol. 178 (1996) 7-16). Other formulations of the disposition function are available (Balaz S., Quant. Struct. Act. Relat. 13 (1994) 381-392) or can be constructed using the principles described therein. In general, the disposition functions are nonlinear in optimized parameters.

A more complete description of biological activity in complex systems thus includes the disposition function as well as the association constant Kᵢ (Eq. (5)). Biological activity is most frequently described as the product of the disposition function and the association constant, but other relationships are possible (Balaz et al., Quant. Struct. Act. Relat. 4 (1985) 77-81).

Mathematical descriptions of biological activity that include both the disposition function and the association constant Kᵢ can be linearized using the Taylor expansion. Due to a more complicated functional form, the simplified approach as described above for the binding function Eq. (5), may not always be applicable and the rigorous approach must be used. For instance, if biological activity BA is expressed as the product of the disposition function A as defined by Eq. (8) and the association constant K as defined by Eq. (5) the expressions for iterative optimization of the regression coefficients C contained in both the functions would be obtained from:

$$\begin{matrix}BA ( C \to {C}^{′} ) = AK ( C = {C}^{′} ) + \\ {( \frac{\partial ( AK )}{\partial A} )}_{C = {C}^{′}} ( A - {A}^{′} ) + {( \frac{\partial ( AK )}{\partial B} )}_{C = {C}^{′}} ( B - {B}^{′} ) + …\end{matrix} \qquad ( 10 )$$

In Eq. (10), the summation goes through all optimized regression coefficients in Eqs. (5) and (8) (i.e. A, B, C, D, A, C₀, Cₑ, all Cₖ). C indicates all the regression coefficients and the primed symbols are either initial estimates or the values of the regression coefficients from the previous iteration. All regression coefficients are simultaneously optimized by PLS. It should be appreciated that the described linearization procedure is not limited to interaction energy and can include the disposition function to describe biological activity in complex systems.

The present invention is illustrated by the following example. It is to be understood that the particular examples, materials, amounts, and procedures are to be interpreted broadly in accordance with the scope and spirit of the invention as set forth herein.

### EXAMPLE 1

### Multi-Mode Binding of Ligands to Receptor Sites: Implementation in CoMFA

Multi-mode Comparative Molecular Field Analysis (CoMFA) was applied to published data for binding of 34 polychlorinated dibenzofurans (PCDF) to the aryl hydrocarbon (Ah) receptor. Several QSAR analyses of these data have already been published (Safe et al., Chemosphere 1985, 14, 675-683; Long et al., Quant. Struct. Act. Relat. 1987, 6, 1-7; Sulea et al., SAR QSAR Environ. Sci. 1995, 3, 37-61; Vedani et al., Altex 1999, 16, 9-14; Mekenyan et al., Environ. Health Perspect. 1996, 104, 1302-1310; and Todeschini et al., Quant. Struct. Act. Relat. 1997, 16, 120-125) including two CoMFA studies (Waller et al., Chem. Res. Toxicol. 1995, 8, 847-858; and Waller et al., J. Med. Chem. 1992, 35, 3660-3666). Surprisingly, the results were rather modest, even though no complicating factors (neither biological such as subcellular distribution, metabolism or different mechanisms leading to biological response, nor chemical such as conformational flexibility or ionization) were encountered. We postulated that a possible reason for the unimpressive results was that the compounds might bind to receptor in several binding modes due to the symmetry of the dibenzofuran skeleton.

Accordingly, the CoMFA procedure was applied to PCDF congeners binding in one, two, four, and 16 modes simultaneously. Descriptive and predictive abilities of the 16-mode model were found to be significantly better than for the one-mode model. Since the two models do not differ in the number of optimized parameters, the improvement is believed to be due to a more realistic description of the binding interactions.

Methods

Studied Data Set. The multi-mode CoMFA was applied to data on binding of a set of polychlorinated dibenzofurans (PCDF) to the aryl hydrocarbon (Ah) receptor. The binding was monitored in a single laboratory as the displacement of radio-labeled 2,3,7,8-tetrachloro dibenzodioxin (TCDD) by PCDF congeners. Ligand binding initiates a sequence of events (including dissociation of the 90 kD heat shock protein) leading to irreversibility of the ligand/receptor interaction. However, the events are rather slow and if PCDF are added simultaneously with TCDD, no irreversibility is observed (Henry, et al., Biochem. J. 1993, 294, 95-101). Binding affinities of 34 PCDF derivatives (Safe et al., Chemosphere 1985, 14, 675-683; Mason et al., Toxicology 1985, 37, 1-12; Safe et al., Environ. Health Perspect. 1985, 60, 47-56; Safe et al., Crit. Rev. Toxicol. 1990, 21, 51-88; and Safe et al., Ann. Rev. Pharmacol. Toxicol. 1986, 26, 371-399) are summarized in Table 1. The complete data set was split into two subsets, training and test set consisting of 22 and 12 derivatives, respectively. Only compounds in the training data set were used for model development. The compounds for the test set were selected so that (i) their binding affinities are evenly distributed within the range of pEC₅₀ values; (ii) all degrees of substitution are included (number of compounds-number of chlorine substituents: 1-0, 1-1, 1-2, 2-3, 3-4, 3-5, and 1-6); (iii) each chlorine position is represented at least once. The test set also contained the congeners 1, 4, and 15 for which only semi-quantitative estimates of binding affinity are available.

Structure Optimization. All molecules were built de novo using the sketch option of Sybyl (version 6.7. Tripos Inc., St. Louis, Mo., USA, 2001). Full geometry optimization and calculation of partial atomic charges was done with GAMESS (Schmidt et al., J. Comp. Chem. 1993, 14, 1347-1363) using ab initio approach with restricted Hartree Fock wavefunction and basis set 6-31 G.

Alignment. The prototypical Ah receptor ligand, TCDD, was chosen as a template for alignment of PCDF molecules. TCDD and PCDF molecules are rigid, planar, and of similar size. All superpositions were constructed in the way that the dibenzodioxin and dibenzofuran skeletons substantially overlapped. In absence of contrary experimental evidence, we assumed that PCDF molecules bind in about the same space as displaced TCDD. This assumption was used as the first choice and since the results were satisfactory, no additional subspaces, neither in directions perpendicular to the TCDD skeleton plane nor reaching far beyond in the plane occupied by TCDD, were explored. The multi-mode CoMFA analyses systematically examined two, four, and sixteen binding modes for each ligand.

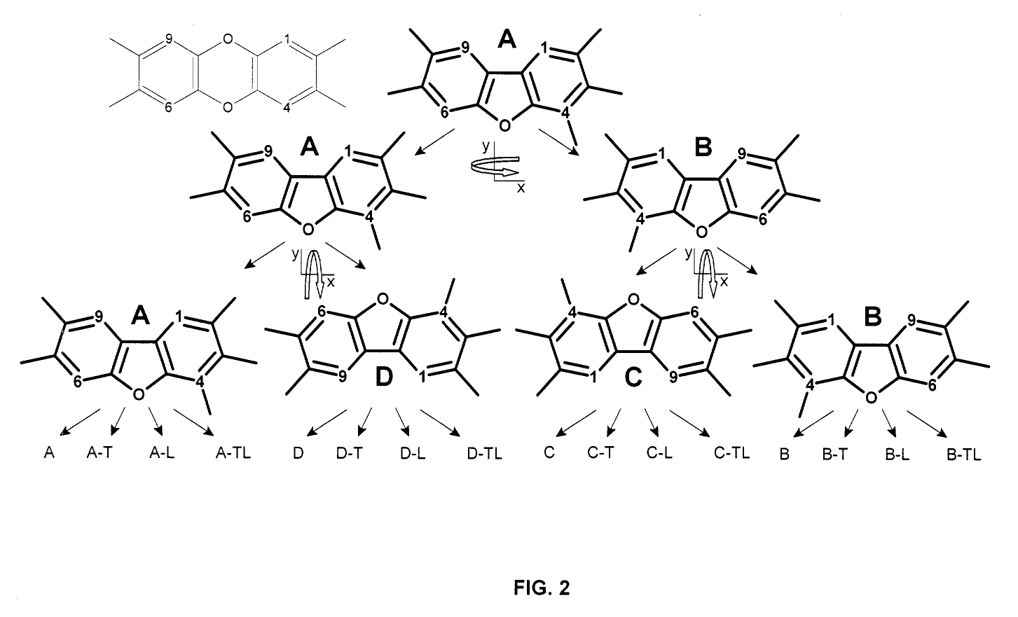

The two and four binding modes were constructed by superposition of 1, 4, 6, and 9 carbon atoms of TCDD and PCDF (FIG. 2). The dibenzofuran skeleton is symmetric around the y axis so the PCDF molecules can bind in the forward mode (A) as well as in the reversed (B) mode. The modes A and B were used in the two-mode CoMFA analysis. The TCDD molecule contains two oxygen atoms while PCDF molecules only have one oxygen. Hypothetically, a PCDF molecule could be oriented in the binding site with oxygen atom in the position opposite to that in modes A and B, in the forward or reverse mode (C and D). The four hypothetical binding modes differ in 180°-rotation of the molecule around the x- or y-axes (FIG. 2). The modes A-D were used in the four-mode CoMFA analysis.

Implementation of binding modes as suggested above assumes strong interactions of aromatic rings or oxygens with the binding site, which would hold skeletons of all PCDF molecules in the same place within the receptor site. If such interactions do not occur, the PCDF molecules could shift from the skeleton-superimposed positions inside the binding site to engage in the same attractive steric interactions as the TCDD molecule. No information on the binding site structure is available, so we decided to roughly describe the shape of the binding cavity as a rectangular box surrounding the TCDD molecule. The focus on the “two-dimensional” PCDF binding in the plane of the TCDD molecule, as described above, eliminates the two walls of the box that are parallel to the skeleton plane. Consideration of four symmetric binding modes allows for further reduction because the placement of boundary walls on opposite sides of the box should provide comparable results.

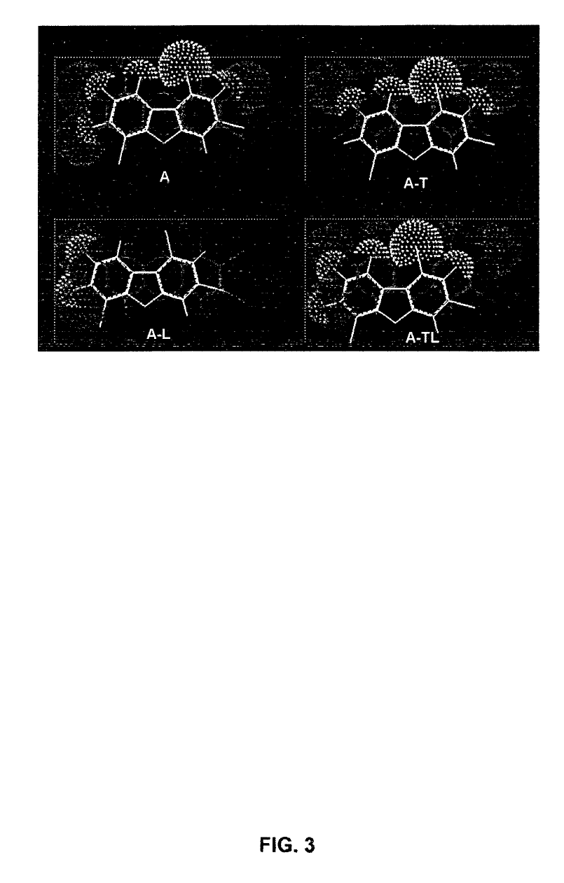

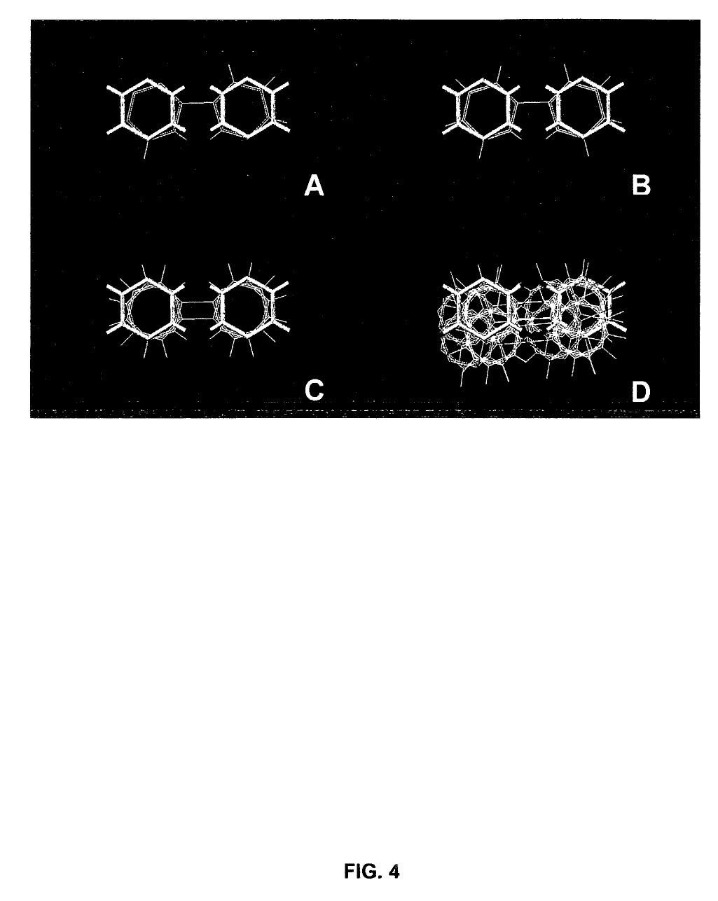

Based on these assumptions, two putative boundary walls of the binding site were set: one on the left side and the other on the upper side of the TCDD molecule. The exact positions of these walls were given by the van der Waals surfaces of chlorines in positions 7 and 8, and hydrogens in positions 1 and 9 of TCDD. From each of the four modes A-D created by the 180°-rotation around x and y axes and superposition of the skeletons, three more modes were formed by translation of the PCDF molecule in the skeleton plane to the left wall, the top wall, and both left and top walls so that the Van der Waals surfaces of the ligand atoms touch the planes (FIG. 3). An illustration of resulting alignments for one, two, four, and sixteen modes is provided in FIG. 4.

CoMFA Interaction Energy Calculations. Steric and electrostatic interaction energies were calculated at each lattice intersection of a regularly spaced rectangular grid (2 A in each coordinate direction). The grid extended approximately 4.0 Å in every direction away from the aligned molecules. The grid's coordinates were: −8 to 8 Å along the x-axis, −6 to 6 A along the y-axis, and −4 to 4 Å along the z-axis, with the center of the TCDD molecule being placed in the origin. An sp³ carbon atom with the charge +1 was used as a probe. The maximum allowable steric and electrostatic energy values were set to 30 kcal/mol. The electrostatic energy term was not calculated in the grid points where the steric energy term reached maximal values (30 kcal/mol). Distance dependent dielectric constant was used.

Variable Pre-selection. The lattice described above consists of 315 grid points leading to 630 descriptor variables—steric and electrostatic interaction energies Xᵢⱼₖ. Most variables were insignificant due to low variation throughout the set of PCDF molecules. Only the variables with the standard deviation SD>3 and sustained variability were selected for optimization.

Using smart region definition (SRD) within GOLPE, version 4.5. Multivariate Infometric Analysis Srl., Perugia, Italy, 1999, the chosen columns were sorted into groups carrying similar information. First, the PCA model with dimensionality five was created. Then SRD was performed in the chemometrical space of PCA loadings. The 31 most informative variables (seeds) were selected and the other variables were assigned to the seeds if their distance from the seed was less than 1.0. Resulting Voronoi polyhedra were merged, if their seeds were closer than 2.0 Å and if they contained the same information as assessed by the average values of all, positive, and negative point energies in the regions (Pearson's R>0.8 for averages of all point energies and R>0.5 for averages of positive and negative point energies).

Some groups contained variables corresponding to grid points symmetrically placed in space. Because the PCDF molecules have a symmetric skeleton and the binding modes were symmetric as well, the points aligned symmetrically are expected to carry similar information. Therefore the symmetric groups were further merged. From these groups, the variables were gradually added to the current correlation equation according to following criteria: (1) grid points corresponding to the chosen variables were evenly distributed around the molecule, (2) all groups were represented more or less equally, (3) both fields were equally represented, but the variables with steric and electrostatic interaction energies might not correspond to the same grid points.

Results and Discussion

Kinetic and thermodynamic analysis of multi-mode ligand binding to one receptor site shows that the overall (observed) association constant is equal to the sum of partial association constants characterizing individual binding modes as given in Eq. (2). This recipe can be used to incorporate multiple binding modes into any 3D-QSAR method. However, it is worth mentioning that Eq. (2) is valid for one molecular species only and cannot be applied to multi-species binding of different protomers or tautomers of a ligand as recently suggested for protomers (Vedani et al., J. Med Chem. 2000, 43, 4416-4427). In such case, Eq. (3) is to be used.

Implementation into CoMFA is complicated by the fact that the dependence of the binding affinity on the steric and electrostatic ligand/probe energies (Eq. (5)) is nonlinear in optimized regression coefficients C. In order to use the PLS optimization procedure, Eq. (5) had to be linearized (Eq. (7)) and applied iteratively.

PCDF Binding to Ah Receptor. The multi-mode CoMFA procedure is demonstrated here using published data on binding of PCDFs (Table 1) to the Ah receptor. Binding affinities of 34 congeners (Table 1) were carefully determined (cf. very low standard errors, where available) in a single laboratory (Safe et al., Crit. Rev. Toxicol. 1990, 21, 51-88) as the displacement of radiolabeled TCDD (see FIG. 2 for structure). PCDF (Table 1, FIG. 2) are completely rigid molecules and differ only in the number and positions of the chlorine substituents. These characteristics make the data set a superb object for 3D-QSAR studies (Vedani et al., Altex 1999, 16, 9-14; Mekenyan et al., Environ. Health Perspect. 1996, 104, 1302-1310; Todeschini et al., Quant. Struct. Act. Relat. 1997, 16, 120-125; Waller et al., Chem. Res. Toxicol. 1995, 8, 847-858; and Waller et al., J, Med. Chem. 1992, 35, 3660-3666). The published results were rather modest, taking into account absence of factors that usually complicate 3D-QSAR analyses, e.g. conformational flexibility and ionization. We decided to examine one of the plausible causes of this behavior that was not considered in previous studies—multiple binding modes of PCDF due to symmetry of their skeleton.

Multi-mode Alignments. Multi-mode binding was systematically analyzed for two, four, and sixteen hypothetical modes of each ligand (FIG. 2). Two and four binding modes were generated by atom-based superposition of the PCDF and TCDD skeletons. Sixteen modes resulted from translation in the skeleton plane of the PCDF ligands in each of the superposition-based four modes to ensure the contact with the left, top, or both left and top walls of putative binding site represented by a box enclosing the TCDD molecule (FIG. 3). A summary of all alignments for one PCDF congener is provided in FIG. 4. For 16 modes, the aligned molecules represent a really complex cluster.

Optimal geometries and charges of the PCDF ligands were calculated using GAMESS (Schmidt et al., J. Comp. Chem. 1993, 14, 1347-1363) with the 6-31G basis set. All other calculations were done in the QSAR module of Sybyl (version 6.7. Tripos Inc., St. Louis, Mo., USA, 2001) with non-standard procedures coded in the Sybyl Programming Language. Electrostatic and steric energies Xᵢⱼₖ of interaction between the ligands and the CH₃⁺ probe were used.

Iterative Optimization. Iterative application of linearized Eq. (7) requires initial estimates of the regression coefficients. Since the high number of variables precludes an exhaustive evaluation of possible initial estimates, we used a forward-selection method (FIG. 1) with gradual addition of variables, which were selected on the basis of high and sustained variability, low collinearity, and even spatial distribution. The SRD procedure within GOLPE (Pastor et al., J. Meld Chem. 1997, 40, 1455-1464) classified the variables into several groups, from which the variables were gradually added to the optimized set of variables so that the grid points corresponding to the chosen variables were evenly distributed around the molecules, and all groups and both fields were represented more or less equally. The optimization process consists of three phases: Outlining, Shaping, and Detailing (FIG. 1).

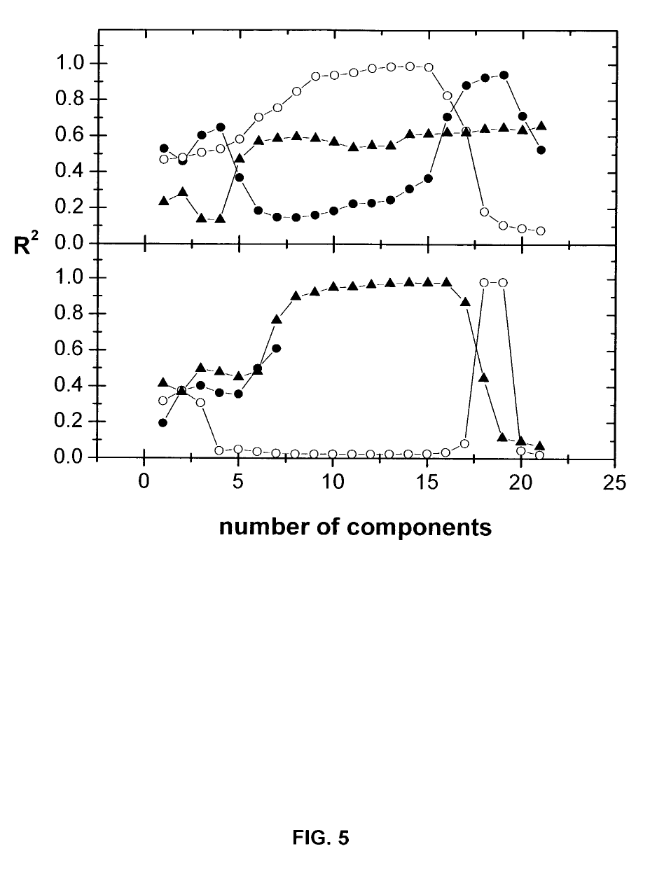

Iterative Procedure. Iterative procedure is the engine of each phase in the optimization process. Its purpose is to optimize the nonlinear regression coefficients C in Eq. (5) by PLS starting with a set of initial estimates. PLS works on equations that are linear in the regression coefficients. For optimization by PLS, Eq. (5) was linearized (Eq. (7)). Each iteration in the Iterative Procedure consists of four steps: (i) setting the values of the regression coefficients C (initial estimates or the set from the last run); (ii) calculation of the partial association constants Kᵢⱼ using Eq. (4); (iii) construction of the coefficients of the linearized Eq. (7) using resulting Kᵢⱼ; and (iv) approximation of the regression coefficients C in Eq. (7) by PLS for all numbers of components. The dependence of the fit quality on the number of components in each PLS run exhibits several extremes with unpredictable positions (FIG. 5). Therefore, in step (iv) of each iteration, the PLS correlation equations for all components, up to the number of included variables or the number of compound in the set (whichever is smaller), had to be enumerated and no extreme-seeking methods (e.g. golden section, Press et al., Numerical Recipes. The Art of Scientific Computing; Cambridge University Press, Cambridge, 1986) could effectively be applied to find the optimal number of components faster and to shorten the computations.

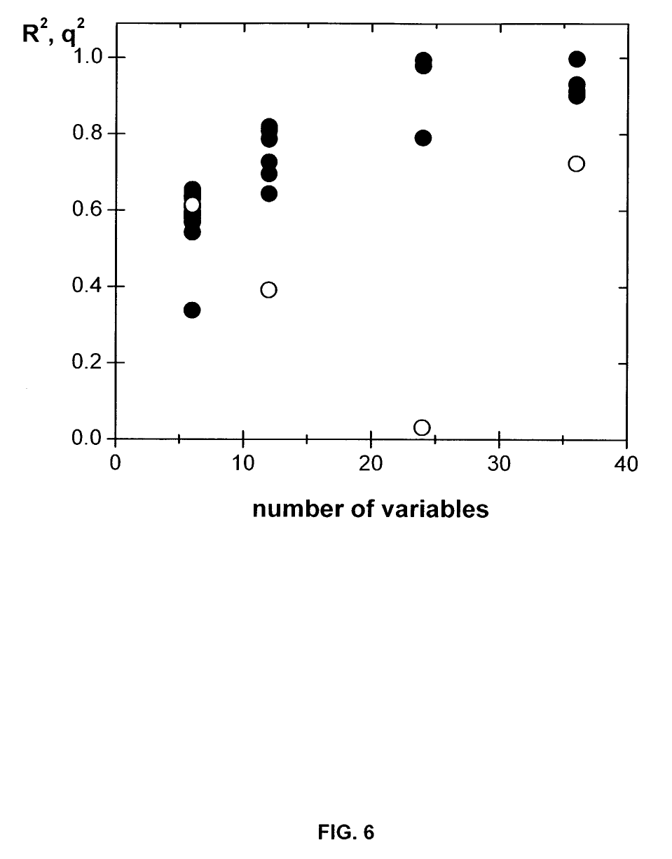

In the first phase of optimization (Outlining, FIG. 1), only six variables (three steric and three electrostatic interaction energies Xᵢⱼₖ) were used. For the sets of regression coefficients C resulting from all plus/minus combinations of numbers 0.1, 0.2, . . . 1.0, the correlations were first calculated using Eq. (5) without optimization. The 15% of the C sets with the lowest SSE were optimized by the Iterative Procedure (a non-linear regression analysis could also have been used here since the number of variables was much lower than the number of compounds). Outlining resulted in the correlations with R² ranging from 0.3 to 0.7 for various considered numbers of modes. The results for 16 binding modes are shown in FIG. 6.

The best 5% of the C sets advanced to the second phase (Shaping, FIG. 1). Here, the group of six new variables was initially added to the best C sets and optimized by the Iterative Procedure. The correlation improved to R² in the range 0.6 to 0.8 (FIG. 6). Further additions of groups of twelve variables led to improvement in correlation to the values of R² between 0.9 and close to 1.0 (FIG. 6). Twenty-four variables were sufficient to obtain a satisfactory correlation for sixteen modes (FIG. 6), as well as for two and four modes (data not shown). However, at least 36 columns (FIG. 6) for 16 binding modes (48 columns for two and four binding modes) were needed to obtain a correlation that would also have good predictive ability. After this point, addition of another 12-variable groups did not significantly improve any statistical parameter. In fact, the predictive correlation coefficient decreased, implying that the new variables (or most of them) brought just more noise to the correlation.

The best correlation equation from the second phase (see FIG. 6 for its statistical indices for 16-mode analysis) was fine-tuned in the third phase of model development (Detailing, FIG. 1). The groups of six or twelve variables, which were rejected in the second phase (Shaping) were broken into smaller sets of one to six variables and tested. For the 2- and 16-mode analyses, no further improvement was reached. For four modes, correlation that could not be further improved by addition of variables contained 52 variables. The Detailing phase (FIG. 1) was finished with the one-by-one reduction of the number of variables. The variables, showing the lowest variability when multiplied by the corresponding regression coefficient C, were omitted.

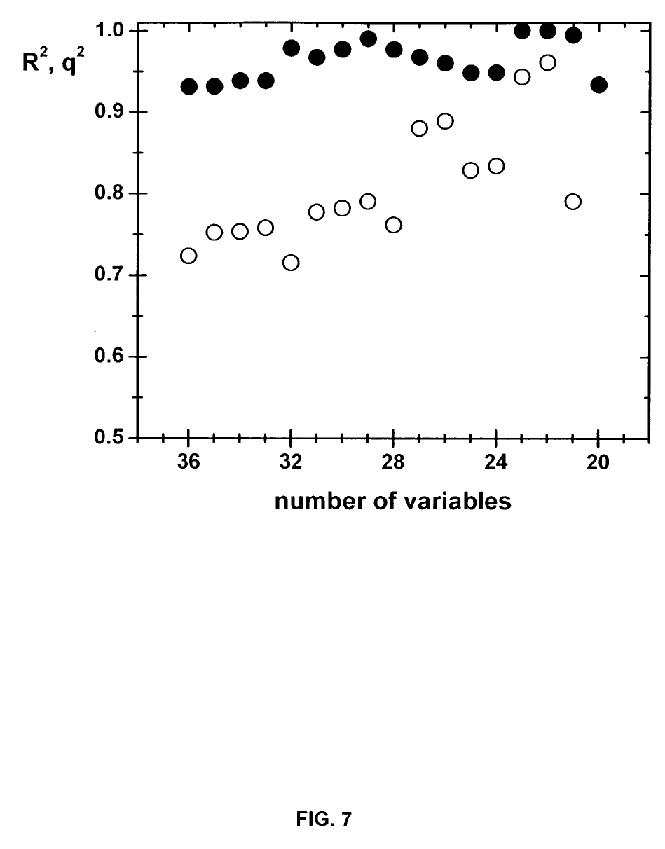

Statistical Evaluation of Mode Additions. Statistical indices for the best correlations for each analyzed number of binding modes are summarized in Table 3. The one-mode CoMFA analysis provided modest results consistent with the published studies. The two- and four-mode correlations show no significant improvement in the predictive ability (SSE and R² for the test set) in comparison with the one-mode correlation. This fact can be explained by inspection of the individual mode prevalencies for the 16-mode model (Table 2). The modes formed by the skeleton alignment (A, B, C, and D) do not contribute to overall binding for any of the compounds in training or testing data set. Incorporation of modes based on putative receptor site significantly improved the fit quality as well as predictive power of the model. These results also suggest that the improvement of the fit quality is not caused by simple addition of more binding modes. If addition of binding modes improved the model just due to increased number of possibilities, the two- and four-mode setups would have to provide better models than one-mode approach. As can be seen in Table 3, this is not the case. The presence of additional binding modes that were only available in the 16-mode setup is crucial for improvement of predictive power. The overall best correlation was produced for 16 starting binding modes for each compound (FIG. 7).

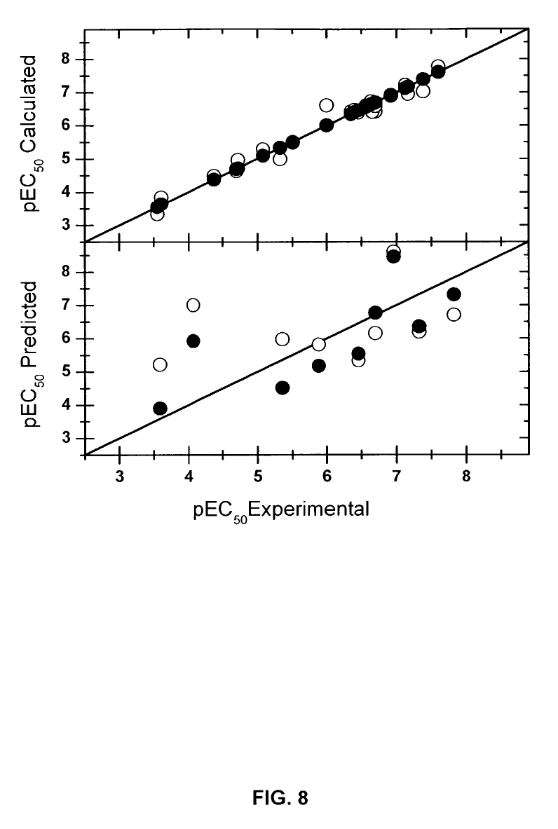

The best multi-mode CoMFA model compares favorably with the one-mode CoMFA as illustrated in Table 3 and FIG. 8. In Table 3, characteristics of the fit quality for CoMFA with weighted contributions of individual modes (current treatment of multiple binding modes in Sybyl (version 6.7. Tripos Inc., St. Louis, Mo., USA, 2001) for the prevalencies of individual modes as determined by the 4-mode correlation on full data set (31 congeners with accurate binding affinities; compounds 1, 4 and 15 with only semiquantitative data were used as a “soft” test set) are also included. All statistical parameters are significantly better for the presented multi-mode procedure. The weighted fields approach, when compared to results of one-mode approach obtained from full data set (data not shown), exhibits better fit (SSE, R²) than the one-mode approach, but is weaker in predictive abilities as indicated by q² and prediction of affinities for the test set compounds 1, 14, and 15 (structures in Table 1) with semi-quantitative pEC₅₀.

It is worth mentioning that the multi-mode approaches do not optimize more regression coefficients C than the one-mode approach. Essentially, the maximum number of coefficients in multi-mode CoMFA is the same as in the standard one-mode approach or the approach with the weighted fields. However, the forward-selection procedure naturally minimizes the number of used variables, so the multi-mode CoMFA actually ends up with fewer optimized coefficients than traditional approaches (e.g., the presented multi-mode correlations only contain ˜5% of variables included in the one-mode correlations).

Optimized Mode Prevalencies. Optimal mode prevalencies are calculated as Kᵢⱼ/Kᵢ=[LRᵢⱼ]/[LRᵢ] (cf. Eq. (2)), whereby Kᵢⱼ are obtained from Eq. (4). The mode prevalence distribution for the 16-mode correlation is summarized in Table 2. Even though 16 modes (FIGS. 2-4) were considered initially, the procedure selected one to four modes that are significantly represented (more than 10%) for each compound.

Most ligands exhibit one binding mode (11 compounds: 1, 2, 4, 6, 10, 13, 15, 16, 18, 21, and 25, Table 2) or two binding modes (18 compounds: 3, 7, 9, 11, 12, 17, 19, 20, 22-24, 26, 28-30, and 32-34). Four congeners (5, 8, 27, and 31) bind in three binding modes and one congener (14) binds in four binding modes. In some cases (marked with superscripts a and b in Table 2), two or four equivalent binding modes were observed and counted as one or two modes. This mode equivalency was caused by symmetrical chlorine positions on the dibenzofuran skeleton. One pair of equivalent modes (for compound 1, Table 2) or four equivalent modes (for compounds 7, 20 and 34) were identified.

Relative Occupancy of Individual Modes. The relative occupancy of individual modes can be estimated by summing up prevalencies of individual modes for all ligands (Table 2) and dividing them by overall occupancy (34, for 34 compounds). The 16 modes can be classified according to increasing occupancy into three groups: A, B, C, D, A-L, and B-L (occupancy less that 0.1%); B-T, B-TL, C-T, C-L, C-TL, and D-TL (0.1-10%); and A-T, A-TL, D-T, and D-L (>10%). Mode C and modes derived from it have the lowest representation in the mode distribution. Only three compounds (1, 3, and 7, Table 2) bind in the four C-related modes in excess of 10% (combined for equivalent modes).

Individual groups of modes exhibit increasing occupancies in the following order: the skeleton-aligned modes A, B, C, D (sum of relative prevalencies =0.12%), the left-aligned modes A-L, B-L, C-L, D-L (22.56%), the top-left aligned modes A-TL, B-TL, C-TL, D-TL (35.59%), and the top-aligned modes A-T, B-T, C-T, D-T (41.73%). The skeleton-aligned modes A-D are practically not represented in the optimized mode distribution. This is an interesting conclusion since the skeleton-based alignments are most frequently used in 3D-QSAR studies.

The left-aligned modes are present mostly for D mode. The only compound that shows significant contribution from other left-aligned mode is compound 7, which is symmetrical and hence the C-L and D-L modes are identical. Interesting observation is that if a single mode contributes to overall binding of a ligand in excess of 90% it is almost exclusively this left-aligned D mode. The exception is compound 13 for which A-T mode contributes 100% to overall binding and compound I that shows only binding in top-aligned mode with oxygen in upward position (symmetrical molecule with binding in C-T and D-T mode).

Binding Site Maps. CoMFA provides a spatial description of the binding site as represented by the differences of interaction fields of ligands between the receptor site and water. The electrostatic and steric maps are formed by significant regression coefficients in considered grid points. The maps generated by the multi-mode approach for the Ah binding site using the PCDF binding data exhibit some peculiarities. Since all ligands are planar and aligned in the plane of the skeleton in all modes, the resulting fields exhibit symmetry about the skeleton plane. The multi-mode approaches analyze the ligand clusters that are essentially symmetrical about the y-axis (two modes) and both x- and y-axes (four and partially in sixteen modes). Consequently, there will be two equivalent resulting maps for the two-mode approach and four equivalent resulting maps for the four-mode and sixteen-mode approaches, all rotated by 180° around the corresponding symmetry axes. The multi-mode approach will arrive at one of the fields depending upon the initial estimates and other details in the numerical procedure.

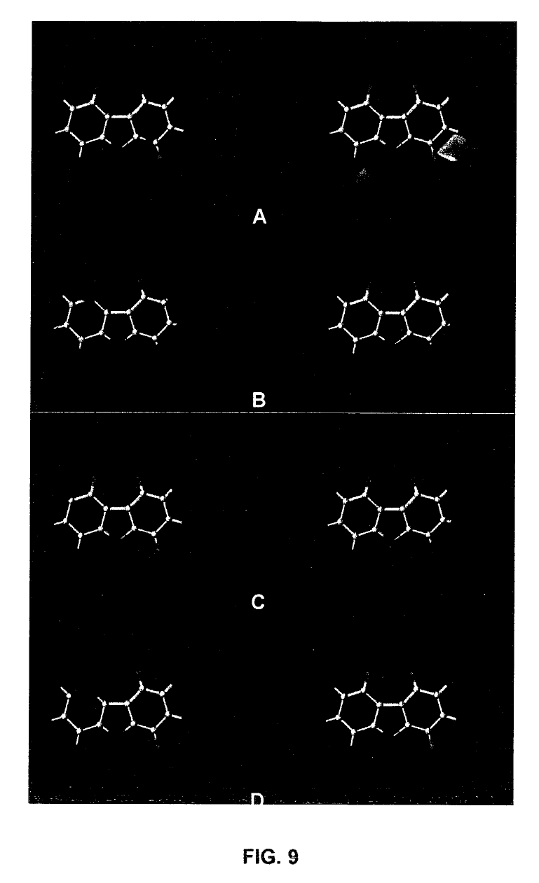

Electrostatic and steric properties of the receptor site as estimated by approaches considering varying number of binding modes are compared in FIG. 9. In general, the main features are preserved in the maps resulting from all approaches. Electrostatic maps show the repulsive regions for positive charge on both sides of the middle part that support binding of the negative TCDD and PCDF oxygens. Electrostatic interactions of chlorines will be weak due to their low charges. Favorable steric regions (green) are localized near the lateral positions on both sides of the molecule. All the features are in accord with the highest binding affinity of TCDD and ligand 29 (Table 2) to the Ah receptor.

Weighted Field Approach. Current version of Sybyl (version 6.7. Tripos Inc., St. Louis, Mo., USA, 2001) treats the problem of multiple binding modes in a completely different way based on the weighted-field approach. It uses standard one-mode procedure, whereby the field representing the multitude of modes is obtained as a weighted average of the fields of individual modes (Cramer III et al., U.S. Pat. No. 5,307,287, 1994). In CoMFA, lnKᵢⱼ of an individual mode is a linear combination of the interaction energies Xᵢⱼₖ as follows from the logarithmized Eq. (4) The overall association constant is correlated as lnKᵢ with the weighted average of individual fields, i.e. lnKᵢ is expressed as the weighted average of lnKᵢⱼ. Taking the anti-logarithm, we get

$${K}_{i} = {K}_{i1}^{{w}_{i}} \times {K}_{i2}^{{w}_{2}} \times … {K}_{im}^{{w}_{m}} = {\prod }_{j = 1}^{m} {K}_{ij}^{{w}_{m}} \qquad ( 11 )$$

where w are the weights of individual modes. Eq. (11) is in contradiction with kinetic and thermodynamic analyses (Jullien et al., J. Chem. Edu. 1998, 75, 194-199; Wang et al., J. Mol. Biol. 1995, 253, 473-492; Balaz et al., Chemometr. Intell. Lab. Sys. 1994, 24, 185-191; Hornak et al., Quant. Struct. Act. Relat. 1998, 17, 427-436; and Smith et al., Chemical Reaction Equilibrium Analysis: Theory and Algorithms; John Wiley and Sons, New York, 1982) that state that the overall association constant is the sum of the partial association constants (Eq. (2)). This discrepancy indicates that the weighted-field approach lacks sound theoretical basis.

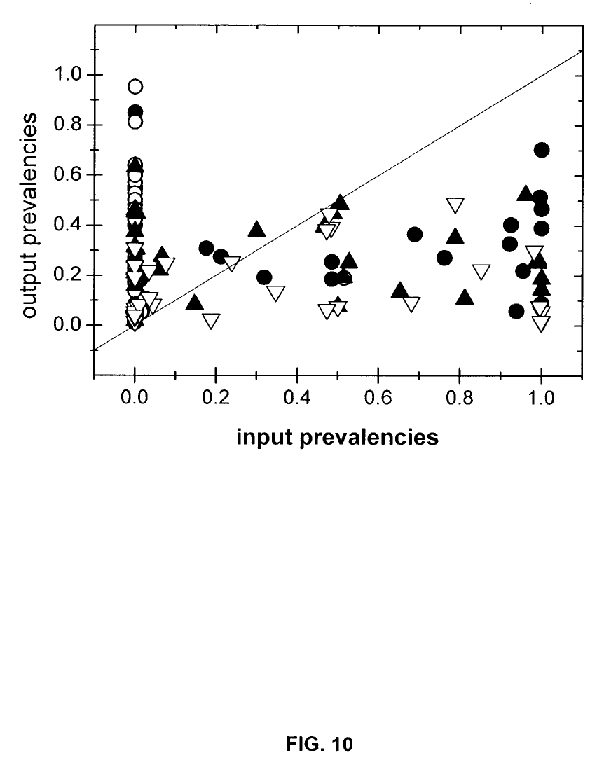

Other examination of the weighted-field approach was done by running the analysis for the data in Table 1. The weighted field values were calculated for the mode prevalencies generated by the four-mode approach. As can be seen in Table 3, the weighted-field approach results in a good fit, but the predictions as determined by the leave-one-out procedure are worse than for the one-mode approach with all compounds in mode A: the values of q² are comparable but the weighted-field approach does not correctly predict the affinity of the compounds 4 and 15 (Table 1) in the test set. Moreover, the input prevalencies of the binding modes as provided by the four-mode approach are not reproduced when calculated back after the analysis using Eq. (4) with optimized regression coefficients Cₖ (FIG. 10). Since the mode prevalencies depend on the optimized regression coefficients, apparently there is no simple scheme to a priori calculate the averaged field that would represent the ensemble of binding modes.

Conclusions and Outlook. The presented multi-mode CoMFA procedure provides statistically better descriptions and predictions than both one-mode and weighted-field approaches. The better predictive ability is not a consequence of increased flexibility of the model due to a higher number of optimized regression coefficients. Just the opposite is true: the used forward-selection approach reduces the number of variables about 20 times as compared with the other approaches.

The multi-mode CoMFA optimizes the prevalencies of individual binding modes. We demonstrated that the procedure effectively selects one to four binding modes out of the ensemble of sixteen modes. This feature makes multi-mode CoMFA a useful tool for selection of binding modes. This ability significantly reduces the subjective input into the alignment procedure and, thus, promotes future automatization of the CoMFA analyses.

Although the current run times are long (the presented results required about 20 days on SGI Octane with 1 R 10000 processor with 250 MHz clock speed, 512 MB RAM), the speed still compares favorably with the number of one-mode analyses which need to be done in order to examine even a small portion of possible combinations of alternate binding modes. The multi-mode procedure can be optimized for faster execution by re-coding the optimization procedure in a language that is faster than SPL as well as by parallelization.

Table 1. Polychlorinated dibenzofurans with binding affinities to the Ah receptor (Safe et al., Crit. Rev. Toxicol. 1990, 21, 51-88). Experimental EC₅₀ values (in mol/L) are given in the form of pEC₅₀=−log(EC₅₀)=logKᵢ. Calculated pEC₅₀ values come from Eq. (5) with optimized regression coefficients. Predicted pEC₅₀ values were obtained for compounds in the test data set (compounds marked with superscript b) from the model derived using the training data set.

| | | pEC₅₀ | | | | |
|---|---|---|---|---|---|---|
| | Cl | | calculated/predicted | | | |
| no. | position(s) | observed^{a} | 1 mode | 2 modes | 4 modes | 16 modes |
| 01^{b} | — | <3.000 | 2.735 | −0.349 | 1.783 | 1.980 |
| 02 | 2 | 3.553 | 3.333 | 3.607 | 3.758 | 3.554 |
| 03 | 3 | 4.377 ± 0.058 | 4.487 | 4.375 | 4.383 | 4.379 |
| 04^{b} | 4 | <3.000 | 3.220 | 1.409 | 1.620 | 2.201 |
| 05 | 2 3 | 5.326 | 4.993 | 4.888 | 5.186 | 5.334 |
| 06 | 2 6 | 3.609 | 3.841 | 3.640 | 3.869 | 3.642 |
| 07^{b} | 2 8 | 3.590 | 5.218 | 4.837 | 4.563 | 3.901 |
| 08^{b} | 1 3 6 | 5.357 | 5.975 | 5.860 | 4.121 | 4.516 |
| 09^{b} | 1 3 8 | 4.071 | 7.005 | 6.891 | 5.688 | 5.923 |
| 10 | 2 3 4 | 4.721 | 4.964 | 4.827 | 4.798 | 4.713 |
| 11 | 2 3 8 | 6.000 ± 0.041 | 6.611 | 6.166 | 6.040 | 6.007 |
| 12 | 2 6 7 | 6.347 | 6.421 | 6.306 | 6.386 | 6.344 |
| 13 | 1 2 3 6 | 6.456 | 6.394 | 6.418 | 6.508 | 6.460 |
| 14^{b} | 1 2 3 7 | 6.959 | 8.598 | 8.584 | 9.570 | 8.458 |
| 15^{b} | 1 2 4 8 | <5.000 | 5.861 | 5.470 | 5.241 | 5.129 |
| 16^{b} | 2 3 4 6 | 6.456 | 5.336 | 5.496 | 4.533 | 5.539 |
| 17 | 2 3 4 7 | 7.602 | 7.773 | 7.717 | 7.661 | 7.609 |
| 18 | 2 3 4 8 | 6.699 | 6.420 | 6.125 | 6.662 | 6.700 |
| 19 | 2 3 6 8 | 6.658 | 6.412 | 6.638 | 6.987 | 6.655 |
| 20 | 2 3 7 8 | 7.387 ± 0.059 | 7.027 | 7.060 | 6.166 | 7.392 |
| 21 | 1 2 3 4 8 | 6.921 | 6.895 | 6.860 | 6.926 | 6.920 |
| 22 | 1 2 3 7 8 | 7.128 ± 0.105 | 7.228 | 7.185 | 7.069 | 7.127 |
| 23 | 1 2 3 7 9 | 6.398 | 6.468 | 6.400 | 6.445 | 6.407 |
| 24 | 1 2 4 6 7 | 7.169 | 6.949 | 7.086 | 7.107 | 7.167 |
| 25 | 1 2 4 6 8 | 5.509 | 5.487 | 5.565 | 5.479 | 5.510 |
| 26^{b} | 1 2 4 7 8 | 5.886 | 5.818 | 5.630 | 5.283 | 5.172 |
| 27 | 1 2 4 7 9 | 4.699 | 4.633 | 4.942 | 4.653 | 4.693 |
| 28 | 1 3 4 7 8 | 6.699 | 6.589 | 6.731 | 6.706 | 6.694 |
| 29^{b} | 2 3 4 7 8 | 7.824 ± 0.028 | 6.706 | 6.671 | 6.356 | 7.314 |
| 30^{b} | 2 3 4 7 9 | 6.699 | 6.155 | 6.682 | 6.653 | 6.770 |
| 31 | 1 2 3 4 7 8 | 6.638 | 6.726 | 6.668 | 6.732 | 6.643 |
| 32 | 1 2 3 6 7 8 | 6.569 ± 0.137 | 6.608 | 6.604 | 6.696 | 6.562 |
| 33 | 1 2 4 6 7 8 | 5.081 | 5.288 | 5.141 | 5.211 | 5.098 |
| 34^{b} | 2 3 4 6 7 8 | 7.328 ± 0.036 | 6.195 | 6.035 | 6.480 | 6.356 |

^{a}When not reported, standard deviation data not available

Table 2. Prevalencies of individual binding modes (FIGS. 2-4) for the 16-mode correlation calculated as Kᵢⱼ/Kᵢ, with the partial association constants Kᵢⱼ obtained from Eq. (4). Structures of ligands are given in Table 1. Significant modes that contributed more than 10% to overall binding are in boldface. Superscripts a and b mark modes that are equivalent due to symmetrical substitution. Superscript c marks compounds that were included in test data set. The last line shows the percentage of all ligands bound in given mode.

| | Prevalence for the mode | | | | | | | | | | | | | | | |
|---|---|---|---|---|---|---|---|---|---|---|---|---|---|---|---|---|
| No | A | A-T | A-L | A-TL | B | B-T | B-L | B-TL | C | C-T | C-L | C-TL | D | D-T | D-L | D-TL |
| 01^{c} | 0.00 | 0.00 | 0.00 | 0.00 | 0.00 | 0.00 | 0.00 | 0.00 | 0.00 | 0.50^{a} | 0.00 | 0.00 | 0.00 | 0.50^{a} | 0.00 | 0.00 |
| 02 | 0.00 | 0.00 | 0.00 | 0.00 | 0.00 | 0.00 | 0.00 | 0.00 | 0.00 | 0.00 | 0.00 | 0.00 | 0.00 | 0.82 | 0.09 | 0.09 |
| 03 | 0.00 | 0.00 | 0.00 | 0.00 | 0.00 | 0.00 | 0.00 | 0.00 | 0.00 | 0.38 | 0.00 | 0.56 | 0.00 | 0.05 | 0.00 | 0.01 |
| 04^{c} | 0.00 | 0.00 | 0.00 | 0.00 | 0.00 | 0.00 | 0.00 | 0.00 | 0.00 | 0.06 | 0.03 | 0.00 | 0.00 | 0.89 | 0.02 | 0.00 |
| 05 | 0.00 | 0.01 | 0.00 | 0.00 | 0.00 | 0.02 | 0.00 | 0.03 | 0.00 | 0.00 | 0.00 | 0.00 | 0.00 | 0.14 | 0.19 | 0.61 |
| 06 | 0.00 | 0.00 | 0.00 | 0.00 | 0.00 | 0.00 | 0.00 | 0.00 | 0.00 | 0.00 | 0.08 | 0.00 | 0.00 | 0.00 | 0.92 | 0.00 |
| 07^{c} | 0.00 | 0.03 | 0.00 | 0.00 | 0.00 | 0.03 | 0.00 | 0.00 | 0.00 | 0.03 | 0.14^{a} | 0.29^{b} | 0.00 | 0.03 | 0.15^{a} | 0.30^{b} |
| 08^{c} | 0.00 | 0.64 | 0.00 | 0.00 | 0.00 | 0.18 | 0.00 | 0.18 | 0.00 | 0.00 | 0.00 | 0.00 | 0.00 | 0.00 | 0.00 | 0.00 |
| 09^{c} | 0.00 | 0.00 | 0.00 | 0.01 | 0.00 | 0.43 | 0.00 | 0.48 | 0.00 | 0.00 | 0.00 | 0.00 | 0.00 | 0.00 | 0.00 | 0.08 |
| 10 | 0.00 | 0.03 | 0.00 | 0.00 | 0.00 | 0.01 | 0.00 | 0.01 | 0.00 | 0.00 | 0.00 | 0.00 | 0.00 | 0.01 | 0.94 | 0.00 |
| 11 | 0.00 | 0.00 | 0.00 | 0.00 | 0.00 | 0.02 | 0.00 | 0.03 | 0.00 | 0.00 | 0.00 | 0.00 | 0.00 | 0.00 | 0.15 | 0.80 |
| 12 | 0.00 | 0.00 | 0.00 | 0.00 | 0.00 | 0.00 | 0.00 | 0.00 | 0.00 | 0.00 | 0.01 | 0.00 | 0.00 | 0.44 | 0.00 | 0.55 |
| 13 | 0.00 | 1.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 14^{c} | 0.00 | 0.30 | 0.00 | 0.33 | 0.00 | 0.00 | 0.00 | 0.00 | 0.00 | 0.00 | 0.00 | 0.00 | 0.00 | 0.14 | 0.00 | 0.23 |
| 15^{c} | 0.00 | 0.08 | 0.00 | 0.00 | 0.00 | 0.00 | 0.00 | 0.00 | 0.00 | 0.00 | 0.00 | 0.00 | 0.00 | 0.00 | 0.92 | 0.00 |
| 16^{c} | 0.00 | 0.00 | 0.00 | 0.00 | 0.00 | 0.00 | 0.00 | 0.00 | 0.00 | 0.00 | 0.00 | 0.00 | 0.00 | 0.01 | 0.99 | 0.00 |
| 17 | 0.00 | 0.01 | 0.00 | 0.01 | 0.00 | 0.00 | 0.00 | 0.00 | 0.00 | 0.00 | 0.00 | 0.00 | 0.00 | 0.51 | 0.00 | 0.47 |
| 18 | 0.00 | 0.02 | 0.00 | 0.00 | 0.00 | 0.01 | 0.00 | 0.01 | 0.00 | 0.00 | 0.00 | 0.00 | 0.00 | 0.00 | 0.96 | 0.00 |
| 19 | 0.00 | 0.00 | 0.00 | 0.00 | 0.00 | 0.49 | 0.00 | 0.46 | 0.00 | 0.00 | 0.00 | 0.00 | 0.00 | 0.00 | 0.05 | 0.00 |
| 20 | 0.00 | 0.22^{a} | 0.00 | 0.27^{b} | 0.00 | 0.23^{a} | 0.00 | 0.28^{b} | 0.00 | 0.00 | 0.00 | 0.00 | 0.00 | 0.00 | 0.00 | 0.00 |
| 21 | 0.00 | 0.01 | 0.00 | 0.00 | 0.00 | 0.00 | 0.00 | 0.00 | 0.00 | 0.00 | 0.00 | 0.00 | 0.00 | 0.00 | 0.99 | 0.00 |
| 22 | 0.00 | 0.41 | 0.00 | 0.45 | 0.00 | 0.03 | 0.00 | 0.03 | 0.00 | 0.00 | 0.00 | 0.00 | 0.00 | 0.03 | 0.00 | 0.05 |
| 23 | 0.00 | 0.46 | 0.00 | 0.52 | 0.00 | 0.01 | 0.00 | 0.01 | 0.00 | 0.00 | 0.00 | 0.00 | 0.00 | 0.00 | 0.00 | 0.00 |
| 24 | 0.00 | 0.57 | 0.00 | 0.43 | 0.00 | 0.00 | 0.00 | 0.00 | 0.00 | 0.00 | 0.00 | 0.00 | 0.00 | 0.00 | 0.00 | 0.00 |
| 25 | 0.00 | 0.02 | 0.00 | 0.00 | 0.00 | 0.00 | 0.00 | 0.00 | 0.00 | 0.00 | 0.00 | 0.00 | 0.00 | 0.00 | 0.98 | 0.00 |
| 26^{c} | 0.00 | 0.45 | 0.00 | 0.48 | 0.00 | 0.04 | 0.00 | 0.03 | 0.00 | 0.00 | 0.00 | 0.00 | 0.00 | 0.00 | 0.00 | 0.00 |
| 27 | 0.00 | 0.41 | 0.00 | 0.40 | 0.00 | 0.18 | 0.00 | 0.01 | 0.00 | 0.00 | 0.00 | 0.00 | 0.00 | 0.00 | 0.00 | 0.00 |
| 28 | 0.00 | 0.00 | 0.00 | 0.01 | 0.00 | 0.53 | 0.00 | 0.46 | 0.00 | 0.00 | 0.00 | 0.00 | 0.00 | 0.00 | 0.00 | 0.00 |
| 29^{c} | 0.00 | 0.45 | 0.00 | 0.51 | 0.00 | 0.02 | 0.00 | 0.02 | 0.00 | 0.00 | 0.00 | 0.00 | 0.00 | 0.00 | 0.00 | 0.00 |
| 30^{c} | 0.00 | 0.49 | 0.00 | 0.49 | 0.00 | 0.01 | 0.00 | 0.01 | 0.00 | 0.00 | 0.00 | 0.00 | 0.00 | 0.00 | 0.00 | 0.00 |
| 31 | 0.00 | 0.14 | 0.00 | 0.67 | 0.00 | 0.10 | 0.00 | 0.09 | 0.00 | 0.00 | 0.00 | 0.00 | 0.00 | 0.00 | 0.00 | 0.00 |
| 32 | 0.00 | 0.46 | 0.00 | 0.42 | 0.00 | 0.06 | 0.00 | 0.06 | 0.00 | 0.00 | 0.00 | 0.00 | 0.00 | 0.00 | 0.00 | 0.00 |
| 33 | 0.02 | 0.50 | 0.02 | 0.42 | 0.00 | 0.02 | 0.00 | 0.00 | 0.00 | 0.00 | 0.02 | 0.00 | 0.00 | 0.00 | 0.00 | 0.00 |
| 34^{c} | 0.01 | 0.26^{a} | 0.01 | 0.22^{b} | 0.01 | 0.26^{a} | 0.01 | 0.22^{b} | 0.00 | 0.00 | 0.00 | 0.00 | 0.00 | 0.00 | 0.00 | 0.00 |
| % | 0.09 | 20.50 | 0.09 | 16.59 | 0.03 | 7.88 | 0.03 | 7.12 | 0.00 | 2.85 | 0.82 | 2.5 | 0.00 | 10.50 | 21.62 | 9.38 |

Table 3: Quality of the CoMFA models considering one binding mode and multiple binding modes treated by the present approach and by the weighted-fields approach (weights obtained by the 4-mode CoMFA on a full set) as implemented in Sybyl (version 6.7. Tripos Inc., St. Louis, Mo., USA, 2001). Structures of the ligands with predicted binding affinities are in Table 1.

| | CoMFA | | | | |
|---|---|---|---|---|---|
| | | | | | weighted |
| | 1 mode | 2 modes | 4 modes | 16 modes | fields |
| No. of variables | 570 | 26 | 22 | 22 | 570 |
| Training set: | | | | | |
| SSE | 1.097 | 0.771 | 1.799 | 0.002 | 2.613 |
| R² | 0.963 | 0.974 | 0.940 | 0.999 | 0.944 |
| PRESS | 6.391 | 2.12 | 2.476 | 1.167 | 16.782 |
| q² | 0.786 | 0.929 | 0.917 | 0.961 | 0.639 |
| Test set: | | | | | |
| SSE | 18.416 | 16.385 | 18.838 | 9.042 | |
| R² | 0.382 | 0.450 | 0.368 | 0.697 | |

Predictions for semi-quantitative data:

| | | | | | |
|---|---|---|---|---|---|
| 1 (<3.000) | 2.735 | −0.349 | 1.783 | 1.980 | 2.609 |
| 4 (<3.000) | 3.220 | 1.409 | 1.620 | 2.201 | 3.445 |
| 15 (<5.000) | 5.861 | 5.470 | 5.241 | 5.129 | 5.230 |

The complete disclosures of all patents, patent applications including provisional patent applications, and publications, and electronically available material cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

What is claimed is:

1. A computer-based method for generating a three-dimensional quantitative structure activity relationship of a series of ligand molecules in association with a common macromolecule, the method comprising:
a. identifying one or more binding modes j for each ligand molecule i in the series of ligand molecules;
b. placing each binding mode j for each ligand molecule i in said series into a grid for calculation of binding energies;
c. determining in a multiplicity of grid points k for each binding mode j of each ligand molecule i, the interaction energy Xᵢⱼₖ of binding mode j with a selected probe;
d. expressing an association constant Kᵢ for each ligand molecule i as a nonlinear function of the interaction energies Xᵢⱼₖ for each binding mode j to yield a nonlinear binding function;
e. optimizing the regression coefficients in the nonlinear binding function;
f. linearizing the nonlinear binding function to allow the use of partial least squares for iterative optimization of at least one regression coefficient to yield a linearized correlation function;
g. applying a partial least squares procedure repetitively to the linearized correlation function until self-consistency to correlate the observed biological activity data with the interaction energies Xᵢⱼₖ of the ligand molecules; and
h. calculating the optimized distribution of prevalencies of individual binding modes using the ratio of partial association constant Kᵢⱼ and the association constant Kᵢ for each ligand molecule i with all the other ligand molecules in the series.

2. The method of claim 1 further comprising visualizing the three-dimensional quantitative structure activity relationship.

3. The method of claim 2 wherein visualizing the three-dimensional quantitative structure activity relationship comprises graphically displaying using computer graphics the correlation among the ligand molecules in said series.

4. The method of claim 1 further comprising, after linearizing the binding function but before applying the partial least squares procedure, linearizing a nonlinear disposition function containing at least one variable describing a property of each ligand molecule i selected from the group consisting of lipophilicity, amphiphilicity, acidity, reactivity, 3-dimensional shape of the ligand molecules, and the time of exposure to the common macromolecule, to allow the use of partial least squares for iterative optimization of at least one regression coefficient to yield a linearized disposition function.

5. The method of claim 4 wherein the applying the partial least squares procedure comprises applying a partial least squares procedure repetitively to the linearized correlation function, the linearized disposition function, and/or a mathematical combination of the linearized correlation and disposition functions until self-consistency to correlate the observed biological activity data with the interaction energies Xᵢⱼₖ and/or properties of the ligand molecules.

6. The method of claim 1 wherein optimizing the regression coefficients comprises employing a strategy selected from the group consisting of forward selection, backward selection and genetic algorithm.

7. The method of claim 1 wherein the binding modes j comprise different conformations or orientations or both.

8. The method of claim 1 wherein at least one ligand molecule comprises a plurality of species that originate by ionization or tautomerism, and where step c comprises determining in a multiplicity of grid points k for each binding mode j for each species of each ligand molecule i, the interaction energy Xᵢⱼₖ of binding mode j for each species with a selected probe.

9. A computer-based method for generating a three-dimensional quantitative structure activity relationship of a series of ligand molecules in association with a common macromolecule, the method comprising:
a. identifying one or more binding modes j for each ligand molecule i in the series of ligand molecules;
b. placing each binding mode j for each ligand molecule i in said series into a grid for calculation of binding energies;
c. determining in a multiplicity of grid points k for each binding mode j of each ligand molecule i, the interaction energy Xᵢⱼₖ of binding mode j with a selected probe;
d. expressing an association constant Kᵢ for each ligand molecule i as a nonlinear function of the interaction energies Xᵢⱼₖ for each binding mode j to yield a nonlinear binding function; and
e. optimizing the regression coefficients in the nonlinear binding function; and
f. calculating the optimized distribution of prevalencies of individual binding modes using the ratio of partial association constant Kᵢⱼ and the association constant Kᵢ for each ligand molecule i with all the other ligand molecules in the series.

10. The method of claim 9 further comprising visualizing the three-dimensional quantitative structure activity relationship.

11. In a method for performing comparative molecular field analysis (CoMFA) of a three-dimensional quantitative structure activity relationship of a series of ligand molecules in association with a common macromolecule, the improvement comprising analyzing one or more binding modes j for each ligand molecule i in the series of ligand molecules by:
a. identifying one or more binding modes j for each ligand molecule i in the series of ligand molecules; b. placing each binding mode j for each ligand molecule i in said series into a grid for calculation of binding energies; c. determining in a multiplicity of grid points k for each binding mode j of each ligand molecule i, the interaction energy Xᵢⱼₖ of binding mode j with a selected probe;
d. expressing an association constant Kᵢ for each ligand molecule i as a nonlinear function of the interaction energies Xᵢⱼₖ for each binding mode j to yield a nonlinear binding function;
e. optimizing the regression coefficients in the nonlinear binding function;
f. linearizing the nonlinear binding function to allow the use of partial least squares for iterative optimization of at least one regression coefficient to yield a linearized correlation function;
g. applying a partial least squares procedure repetitively to the linearized correlation function until self-consistency to correlate the observed biological activity data with the interaction energies Xᵢⱼₖ of the ligand molecules; and
h. calculating the optimized distribution of prevalencies of individual binding modes using the ratio of partial association constant Kᵢⱼ and the association constant Kᵢ for each ligand molecule i with all the other ligand molecules in the series.

12. The method of claim 11 further comprising visualizing the three-dimensional quantitative structure activity relationship.
